(19) United States
(12) Patent Application Publication
(10) Pub. No.: US 2022/0143226 A1
(43) Pub. Date: May 12, 2022

(54) USE OF TRIAZOLO[4,5-D]PYRIMIDINE DERIVATIVES

(71) Applicants: UNIVERSITÉ DE LIÈGE, Liège (BE); CENTRE HOSPITALIER UNIVERSITAIRE DE LIÈGE, Liège (BE)

(72) Inventors: Cécile Oury, Liège (BE); Patrizio Lancellotti, Liège (BE); Alain Nchimi, Liège (BE); André Luxen, Liège (BE); Eric Goffin, Liège (BE); Adrien Stouse, Liège (BE)

(21) Appl. No.: 17/582,283

(22) Filed: Jan. 24, 2022

Related U.S. Application Data
(63) Continuation-in-part of application No. PCT/EP2020/070721, filed on Jul. 22, 2020.

(30) Foreign Application Priority Data
Jul. 25, 2019 (GB) 1910656.6

(51) Int. Cl. A61K 51/04

(52) U.S. Cl. CPC A61K 51/0459

(57) ABSTRACT

A method of imaging a bacterial infection in a host mammal using Triazolo[4,5-d]pyrimidine derivatives of formula (I):

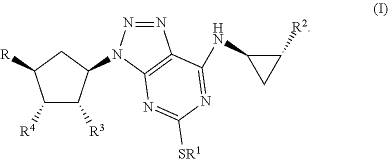

Also disclosed are compositions including the triazolo[4,5-d]pyrimidine derivative.

### FIELD OF THE INVENTION

The present invention relates to a triazolo[4,5-d]pyrimidine derivative for use in prognosis and/or diagnosis in-vivo of a bacterial infection in a host mammal, particularly a deep-seated bacterial infection. The present invention also relates to a method of imaging said bacterial infection and to the use of triazolo[4,5-d]pyrimidine derivative in prognosis and/or diagnosis in-vitro of a bacterial infection.

### INTRODUCTION

Bacterial infection is a major cause of morbidity and mortality, particularly when there is a deep-seated infection.

Deep-seated infections are difficult to diagnose from other causes of inflammation. Current practice relies on biopsy, blood, urine sample analysis or radiological technics such as for example magnetic resonance imaging (MRI), X-Ray, ultrasound (US), X-ray computerized tomography (CT) and the like that allow the infection to be localized and detect morphological changes related to the infection, the host reaction or both. Unfortunately such morphological changes may not be different from inflammation or cancer tumors. Moreover these morphological changes may not be detectable in the early stages of an infection, and remain unspecific when they are present.

Deep-seated infection may also been identified using a tracer labeled with a radioelement and is then called a radiomarker. The radiomarker also called radiotracer can be detected by nuclear imaging techniques such as on single-photon Emission Computer Tomography (SPECT) or positron emission tomography (PET). Examples of radiotracers are ¹⁸F-fluorodeoxyglucose PET, ⁶⁷Ga-citrate SPECT or radiolabeled leukocyte SPECT but they are not specific to bacterial infection and cannot distinguish infection from sterile inflammation or cancer.

There is therefore an urgent need in the art for a new tracer for use in prognosis or diagnosis of bacterial infection, particularly deep-seated infection in a host mammal. There is a need for a tracer that would be bacteria specific and sensitive but also non-toxic, affordable, widely available and easily and rapidly prepared.

### SUMMARY OF THE INVENTION

We have surprisingly found that triazolo[4,5-d]pyrimidine derivatives or a composition thereof can be used as a tracer in the prognosis and/or diagnosis of bacterial infection in a host mammal. Triazolo[4,5-d]pyrimidine derivatives have the advantage to rapidly and non-invasively target early infection and therefore to discriminate between infection and sterile inflammation or cancer. The triazolo[4,5-d]pyrimidine derivatives have also the advantage to diagnose or prognose a large spectrum of bacteria belonging to Gram-positive or Gram-negative species.

The triazolo[4,5-d]pyrimidine derivatives can be absorbed by the bacterial cell and can therefore be used as a tracer for in-vivo or in-vitro prognosis and/or diagnosis of bacterial infection.

### BRIEF DESCRIPTION OF THE DRAWINGS

The invention will now be illustrated with reference to the following Figures of the accompanying drawings which are not intended to limit the scope of the claimed invention:

FIG. 1 shows a reaction scheme illustrating the synthesis of a labelling precursor detailed in step (i) of Example 1; and

FIG. 2 shows a reaction scheme illustrating the synthesis of further labelling precursors detailed in step (ii) of Example 1.

FIG. 3 shows a reaction scheme illustrating the preferred mode of synthesis of a labelling precursor detailed in Example 6 and its conversion into ¹⁸F-Triafluocyl.

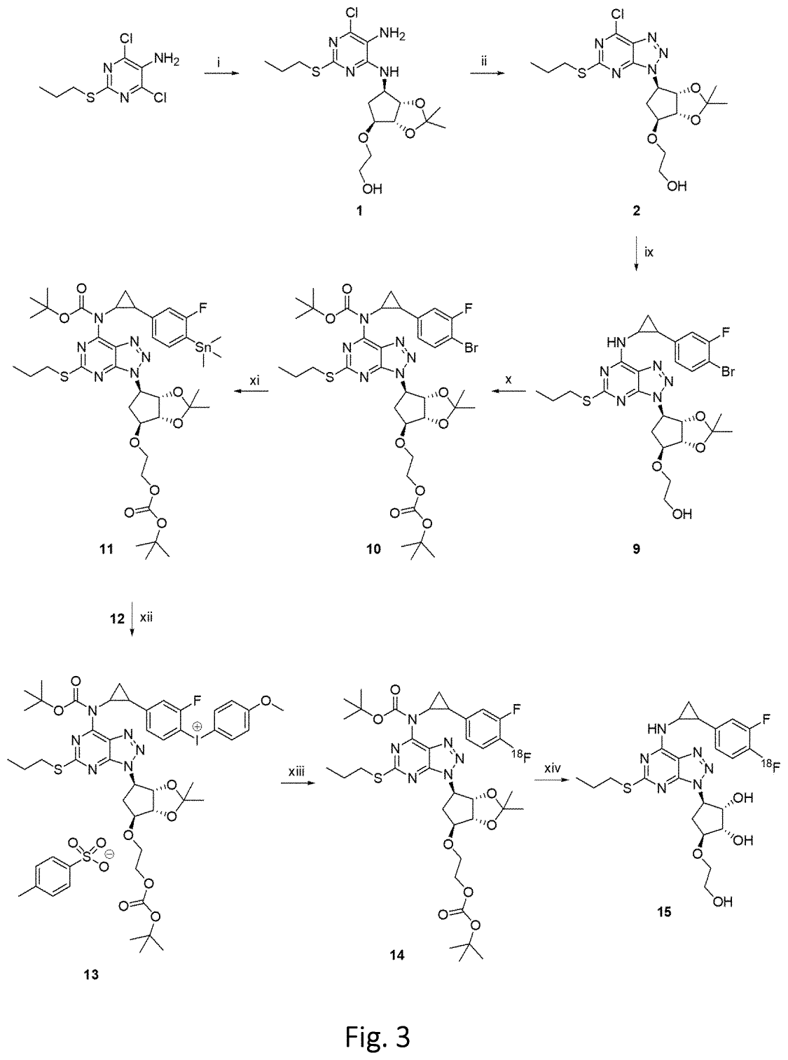

FIG. 4 shows a reaction scheme illustrating the synthesis of another labelling precursor (25) detailed in Example 7 and its conversion into ¹⁸_{F}-(₂₇)_{.}

### DETAILED DESCRIPTION

In one aspect, the invention provides a triazolo[4,5-d]pyrimidine derivative of formula (I) for use in prognosis and/or diagnosis in-vivo of bacterial infection in a host mammal.

In another aspect the invention provides the use of triazolo[4,5-d]pyrimidine derivative of formula (I) in in-vitro prognosis and/or diagnosis of bacterial infection.

The term “bacterial infection” refers for example to pneumonia, septicemia, endocarditis, osteomyelitis, meningitis, urinary tract, skin, and soft tissue infections, but also to cardiac-implant-related infective endocarditis, to prosthetic valve endocarditis or periprosthetic joint infection that occurs in 1 or 2 percent of joint replacement surgeries.

The bacterial infection may be caused for example by one or more of S. aureus, S. epidermidis, E. faecalis, E. faecium, methicillin-resistant S. aureus (MRSA), methicillin-resistant S. epidermidis (MRSE), glycopeptide intermediate S. aureus (GISA), Coagulase-negative staphylococci (CoNS), Vancomycin-resistant enterococci (VRE), beta-hemolytic Streptococcus agalactiae (Group B Streptococcus, GBS) or other streptococci that belong to Gram-Positive bacteria or by one or more of Acinetobacter baumannil, Pseudomonas aeruginosa, carbapenem-resistant Pseudomonas aeruginosa, Enterobacteriaceae, and 3^{rd} generation cephalosporin-resistant Enterobacteriaceae (Klebsiella pneumonia, Escherichia coli, Enterobacter spp, Serratia spp, Proteus spp, Providentia spp, and Morganella spp) that belong to Gram-Negative bacteria.

The term diagnosis as used herein refers to identifying a bacterial infection in the host mammal.

The term prognosis as used herein refers to determining intensity of a bacterial infection in the host mammal at any stage, particularly at an early stage.

The term host mammal as used herein refers preferably to a human but also to an animal.

In a particular embodiment the triazolo[4,5-d]pyrimidine derivative of formula (I) comprises a detectable marker.

In another particular embodiment the triazolo[4,5-d]pyrimidine derivative of formula (I) is bound to a detectable marker.

The term “detectable marker” as used herein refers to any type of tag, which is detectable and thus allows the determination of the presence of the triazolo[4,5-d]pyrimidine derivative:
either directly for example as a radiolabeled triazolo[4,5-d]pyrimidine derivative wherein for example one atom of the derivative has been replaced by one radioisotope marker selected from the group ²H, ³H, ¹³F^{, 18}F, ¹⁹F, ¹¹C, ¹³C, ¹⁴C, ⁷⁵Br, ⁷⁶Br, ¹²⁰I, ¹²³I, ¹²⁴I, ¹²⁵I, ¹³¹I, ¹⁵O, ¹³N, ⁷⁸Br and the like; preferably one halogen atom is replaced by one radioisotope selected from ¹³F, ¹⁸F, ¹⁹F, ⁷⁵Br, ⁷⁶Br, ¹²⁰I, ¹²³I, ¹²⁴I^{, 125}I, ¹³¹I, ⁷⁸Br and the like;
or directly as a radiolabeled triazolo[4,5-d]pyrimidine derivative wherein the derivative may be complexed with a radiomarker such as ⁹⁹Tc, ⁶³Ga, ⁶⁷Ga, ¹¹¹In and the like;
or indirectly for example via a transporter comprising a signal amplifier. The triazolo[4,5-d]pyrimidine derivative is then chemically bound to the transporter and the derivative may be considered as a sensor of bacterial infection.

The term of “signal amplifier” as used herein refers to any agent that would amplify a return on signal at a bacterial infected tissue or implant.

The signal amplifier may for example enhance reflection, refraction, scattering, transmission or attenuation of an ultrasound wave in a US imaging tomography at a bacterial infected tissue or implant; or enhance a resonance or different hydrogen alignment in reaction to a radiofrequency pulse emitted by a magnetic field in magnetic resonance imaging at a bacterial infected tissue or implant; or enhance a proportion of X-ray absorbed or scattered at the bacterial infected tissue or implant in X-Ray computed tomography (CT).

The term “transporter” as used herein refers for example to a body which acts as a signal amplifier or into which the signal amplifier may be incorporated, such as a micelle, microsphere, liposome, polymeric particle, nanosphere, nanosuspension, nano emulsion, nanocapsule and the like.

The triazolo[4,5-d]pyrimidine derivative that comprises a detectable marker via a radioisotope or is bound to a detectable marker either via a complex with the detectable marker or via a transporter comprising the detectable marker is also called hereafter a detectable triazolo[4,5-d]pyrimidine derivative.

In a particular embodiment, the detectable marker is a signal amplifier that is indirectly associated to the triazolo[4,5-d]pyrimidine derivative to be used in a detection method that will depend on the nature of the marker.

The triazolo[4,5-d]pyrimidine derivative may be associated with a signal amplifier such as for example gadolinium (e.g. ⁶⁴Gd) chelates that are ferromagnetic compounds able to enhance magnetic resonance imaging. Gd chelates can be an ionic (e.g. meglumine or sodium salt) or non-ionic signal amplifier. Iron oxide may also be used as signal amplifier for MRI. For example, Manganese doped superparamagnetic iron oxide nanoparticle may be used to form ultrasensitive MRI contrasts agents. Such Mn-SPIO nanoparticles are then self-assembled into clusters inside micelles that will be detectable by MRI. Other signal amplifiers for

MRI are for example a manganese chelate, iron platinum (FePt) alloy nanocrystal, manganese ferrite (MnO—Fe₂O₃) nanocrystal, or other metal-doped iron oxide nanoparticle such as Co—Fe₂O₃ and NiO—Fe₂O₃.

The triazolo[4,5-d]pyrimidine derivative may also be associated with a signal amplifier such as a microbubble to be used in contrast-enhanced ultrasound imaging (US).

The triazolo[4,5-d]pyrimidine derivative may also be associated with a signal amplifier also called contrast agent containing Iodine or Barium for X-Ray computed tomography (CT). The contrast agent should increase the absolute CT attenuation difference between the target bacterial infection and the surrounding tissue. Examples of a suitable contrast agent for X-Ray computer tomography are iohexol (Omnipaque™, GE Healthcare); iopromide (Ultravist™, Bayer Healthcare); iodixanol (Visipaque™, GE Healthcare); ioxaglate (Hexabrix™, Mallinckrodt Imaging); iothalamate (Cysto-Conray II™, Mallinckrodt Imaging); nd iopamidol (Isovue™, Bracco Imaging).

In another particular embodiment, the detectable marker is an isotope which allows the use of the triazolo[4,5-d]pyrimidine derivative as a tracer in a detection method that will depend on the nature of the marker. Accordingly, the triazolo[4,5-d]pyrimidine derivative comprising at least one detectable isotope can be detected by using beta, gamma, positron or x-ray imaging wherein, for example beta or gamma irradiation is provided by the relevant isotope and is detected at an appropriate wavelength.

The triazolo[4,5-d]pyrimidine derivative comprising at least one detectable isotope may be used for example with magnetic resonance spectroscopy (MRS) or imaging (MRI), X-Ray computed tomography (CT), positron emission tomography (PET) and single emission computed tomography (SPECT).

The detectable triazolo[4,5-d]pyrimidine derivative may be detected through isotope ¹⁹F or ¹³C or a combination thereof for MRS/MRI by well-known organic chemistry techniques.

Other detectable triazolo[4,5-d]pyrimidine derivatives may also be detected by isotope selected from ¹⁹F, ¹¹C, ⁷⁵Br, ⁷⁶Br or ¹²⁰I or a combination thereof for PET techniques.

Other detectable triazolo[4,5-d]pyrimidine derivatives may also be detected by an isotope selected from ¹⁸F or ¹¹C or a combination thereof for PET in-vivo imaging and may be prepared as described in Bengt Langström in Acta Chemica Scandinavia, 53:651-669 (1999) or the journal of Nuclear Medicine 58(7): 1094-1099(2017) A. M. J. Paans in https://cds.cern.ch/record/1005065/files/p363.pdf

Other detectable triazolo[4,5-d]pyrimidine derivatives may be detected by ¹²³I and ¹³¹I for SPECT imaging and may be prepared as described by Kulkarni, Int.J.Rad.Appl.& Inst (partB)18:647(1991).

Other detectable triazolo[4,5-d]pyrimidine derivatives may also be detected with technetium-99m(^{99m}Tc), ¹²³I and ¹¹¹IN for SPECT imaging. The triazolo[4,5-d]pyrimidine derivative that is radiolabeled accordingly may be easily prepared by a man skilled in the art by techniques well known in the art and described by Zhuang in Nuclear Medicine & Biology 26(2):217-24 (1999) or by Kulkarni in Nuclear Medicine & Biology 18(6):647-654 (1991) or in technical reports 466 published by the International Atomic Energy Agency in 2008.

The triazolo[4,5-d]pyrimidine derivative wherein one or more atoms are replaced by a radionuclide or isotope may be used as a radiotracer to test cells, tissues or fluids from a host mammal and identify the presence and importance of a bacterial infection in the host for example at the surface of a prosthetic valve.

The term “host mammal”, as used herein refers preferably to a human, but also to an animal.

The term triazolo[4,5-d]pyrimidine derivative refers to a compound of the following formula (I)

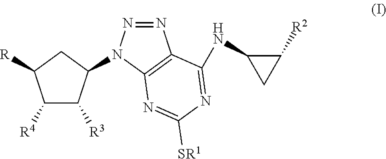

wherein R¹ is C₃₋₅ alkyl optionally substituted by one or more halogen atoms; R² is a phenyl group, optionally substituted by one or more halogen atoms; R³ and R⁴ are each hydroxyl; R is XOH, where X is CH₂, OCH₂CH₂, or a bond;

or a pharmaceutical acceptable salt or solvate thereof, or a solvate thereof or a solvate of such a salt provided that when X is CH₂ or a bond, R¹ is not propyl; when X is CH₂ and R¹ is CH₂CH₂CF3, butyl or pentyl, the phenyl group at R² must be substituted by fluorine; when X is OCH₂CH₂ and R¹ is propyl, the phenyl group at R² must be substituted by fluorine.

Alkyl groups whether alone or as part of another group are straight chained and fully saturated.

In some embodiments, R¹ may represent a C₃₋₅ alkyl optionally substituted by one or more fluorine atoms. Preferably R¹ is 3,3,3-trifluoropropyl, butyl or propyl.

In some embodiments, R² may represent phenyl or phenyl substituted by one or more halogen atoms. Preferably R² is phenyl substituted by one or more fluorine atoms. Most preferably R² is 4-fluorophenyl or 3,4-difluorophenyl.

In some embodiments, R may represent XOH, where X is CH₂, OCH₂CH₂, or a bond; preferably R is OH or OCH₂CH₂OH.

Most preferred triazolo[4,5-d]pyrimidine derivatives are compounds of formula (I) where R² represents 4-fluorophenyl or 3,4-difluorophenyl and/or where R represents OCH₂ CH₂OH.

Triazolo[4,5-d]pyrimidine derivatives are well-known compounds. They may be obtained according to the method described in U.S. Pat. No. 6,525,060 which is described at column 3 line 26 to column 8 line 14 which is incorporated herein by reference.

Prefered triazolo[4,5-d]pyrimidine derivatives are derivatives where R represents OH or OCH₂CH₂OH and/or R² represents 4-fluorophenyl or 3,4-difluorophenyl.

Most preferred triazolo[4,5-d]pyrimidine derivatives are: (1R-(1α, 2α, 3β(1R*, 2*),5β))-3-(7-((2-(3,4-difluorophenyl)cyclopropyl)amino)-5-((3,3,3-trifluoropropyl)thio)-3H-1,2,3-triazolo[4,5-d]pyrimidin-3-yl)-5-(hydroxy)cyclopentane-1,2-diol;

(1S-(1α, 2α, 3β(1R*, 2*),5β))-3-(7-((2-(3,4-difluorophenyl)cyclopropyl)amino)-5-(propylthio)-3H-1,2,3-triazolo[4,5-d]pyrimidin-3-yl)-5-(2-hydroxyethoxy)cyclopentane-1,2-diol;

(1S,2S,3R,5S)-3-[7-[(1R,2S)-2-(3,4-difluorophenyl)cyclopropylamino]-5-(propylthio)-3H-[1,2,3]triazolo[4,5-d]pyrimidin-3-yl]-5-(2-hydroxyethoxy)-1,2-cyclopentanediol;

(1S,2S,3R,5S)-3-[7-[(1R,2S)-2-(4-fluorophenyl)cyclopropylamino]-5-(propylthio)-3H-[1,2,3]triazolo[4,5-d]pyrimidin-3-yl]-5-(2-hydroxyethoxy)-1,2-cyclopentanediol)

or a pharmaceutical acceptable salt or solvate thereof, or a solvate thereof or a solvate of such a salt.

The most preferred triazolo[4,5-d]pyrimidine derivative to be used in prognosis and/or diagnosis is (1S,2S,3R,5S)-3-[7-[(1R,2S)-2-(3,4-difluorophenyl)cyclopropylamino]-5-(propylthio)-3H-[1,2,3]triazolo[4,5-d]pyrimidin-3-yl]-5-(2-hydroxyethoxy)-1,2-cyclopentanediol) as defined in formula (II) (and also called Triafluocyl hereafter):

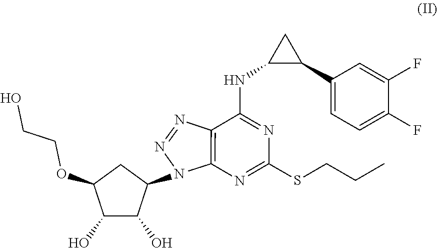 [SMILES: CCCSc1nc(N[C@@H]2C[C@H]2c2ccc(F)c(F)c2)c2nnn([C@@H]3C[C@H](OCCO)[C@@H](O)[C@H]3O)c2n1]

or a pharmaceutical acceptable salt or solvate thereof, or a solvate thereof or a solvate of such a salt.

Another most preferred triazolo[4,5-d]pyrimidine derivative to be used in prognosis and/or diagnosis is (1S,2R,3S,4R)-4-[7-[[(1R,2S)-2-(3,4-difluorophenyl)cyclopropyl]amino]-5-(propylthio)-3H-1,2,3-triazolo[4,5-d]pyrimidin-3-yl]-1,2,3-cyclopentanetriol as defined in formula (III) (and also called Fluometacyl):

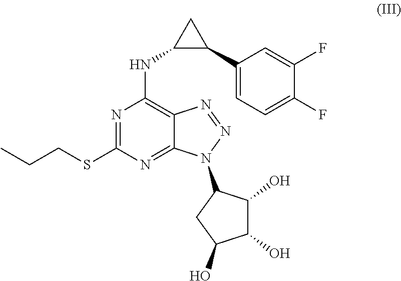

or a pharmaceutical acceptable salt or solvate thereof, or a solvate thereof or a solvate of such a salt.

In some embodiment, one atom of the triazolo[4,5-d]pyrimidine derivative of formula (I) is selected from the group consisting of ³H, ¹³F^{,18}F, ¹⁹F, ¹¹C, ¹³C, ¹⁴C, ⁷⁵Br, ⁷⁶Br, ¹²⁰I, ¹²³I, ¹²⁴I, ¹²⁵I, ¹³¹I, ¹⁵O, ¹³N, ⁷⁸Br.

In some embodiment, one halogen substituent of the triazolo[4,5-d]pyrimidine derivative of formula (I) may be ¹⁸F or ¹²³I.

In some embodiment, one halogen substituent of the Triazolo(4,5-d)pyrimidine derivative of formula (I) may be ¹⁸F.

In some embodiment, one halogen substituent of R² in the triazolo[4,5-d]pyrimidine derivative of formula (I) may be ¹⁸F.

In a further aspect, the present invention provides a pharmaceutical composition comprising the triazolo[4,5-d]pyrimidine derivative of formula (I) thereof and a pharmaceutically acceptable additive, for use in diagnosing and/or prognosing in-vivo bacterial infection in a host mammal;

In a still further aspect, the present invention provides the use of a pharmaceutical composition comprising the triazolo[4,5-d]pyrimidine derivative of formula (I) thereof and a pharmaceutically acceptable additive, in in-vitro prognosis and/or diagnosis of bacterial infection.

The pharmaceutical composition may be a dry powder or a liquid composition having physiological compatibility. In some embodiments, the pharmaceutically acceptable additive may be an auxiliary substance, preservative, solvent and/or viscosity modulating agent.

By solvent, is meant any suitable physiologically compatible solvent such as for example water, saline or any other physiological solution, ethanol, glycerol, oil such as vegetable oil or a mixture thereof. By viscosity modulating agent is meant for example sugar polymer such as carboxymethylcellulose, polysaccharide such as saccharine, and the like.

In some embodiments, the triazolo[4,5-d]pyrimidine derivative or pharmaceutical composition thereof comprising a detectable marker and used as radiotracer may be administered locally or systemically by inhalation, ingestion or injection at a dose that is relevant to a selected imaging device. The administration may be orally, parenterally, topically, rectally, nasally, vaginally.

By parenterally is meant subcutaneously, intravenously, intraarterially, intraperitoneally, intrathecally, intraventricularly and the like.

Dose levels of administration to the host mammal depend upon its age, weight, general health, sex, time of administration, form of administration and the like and would be well known by the one skilled in the art. They may vary between 0.001 μg/kg/day and 10,000 mg/kg/day according to the imaging technique selected.

In other embodiments, the triazolo[4,5-d]pyrimidine derivative or pharmaceutical composition thereof comprising a detectable marker and used as radiotracer may be added to a sample obtained from the host mammal, in an effective amount that is relevant to a selected imaging device.

By sample obtained from the host mammal is meant any sampling of cells, tissues, or body fluids, in which bacterial infection can be determined. Examples of such samples include blood, lymph, urine, biopsies, or bone marrow.

Sample may also refers to an implant, the surface on which bacterial infection can be determined.

By implant, one means all implantable foreign material for clinical use in host mammals such as for prosthetic joints, pacemakers, implantable cardioverter-defibrillators, intravascular or urinary catheters, stent including coronary stent, prosthetic heart valves, intraocular lens, dental implants and the like.

In still another aspect, the present invention also provides a method of imaging a bacterial infection in a host mammal which method comprises a step of administering a detectable amount of triazolo[4,5-d]pyrimidine derivative to the host mammal.

In a further aspect, the present invention also provides the use of triazolo[4,5-d]pyrimidine derivatives in an in-vitro method of imaging a bacterial infection.

In some embodiments, the method of imaging may comprise the following steps:

(a) administering to the host mammal a detectable amount of triazolo[4,5-d]pyrimidine derivative of formula (1) comprising a detectable marker as described above or of the pharmaceutical composition; or adding to a sample obtained from the host mammal a detectable amount of triazolo[4,5-d]pyrimidine derivative of formula (1) comprising a detectable marker as described above or of the pharmaceutical composition; and

(b) tracking said detectable triazolo[4,5-d]pyrimidine derivative by an imaging technique such as for example Magnetic Resonance imaging (MRI), single-photon emission computer tomography (SPECT), positron emission tomography imaging (PET), positron emission tomography with computer tomography imaging, positron emission tomography with magnetic resonance imaging;

and displaying an image of said bacterial infection.

In a particular embodiment the imaging technique is positron emission tomography PET or Single Photon Emission Computed Tomography (SPECT).

The use of a detectable triazolo[4,5-d]pyrimidine derivatives of formula (I) or composition thereof is useful for displaying bacterial infection but also to monitor treatment of bacterial infection in a host. Indeed, with a better knowledge of the bacterial infection severity, it is possible to better select an appropriate treatment and to reduce a potential development of bacterial resistance.

If a detectable triazolo[4,5-d]pyrimidine derivatives of formula (I) or composition thereof is administered or added to a sample obtained from the host before treatment of the infection (e.g. by administration of an antibiotic), it will be possible to administer the right effective amount of antibiotic to the host.

In still another aspect, the present invention also provides a tracer, preferably a positron emission tomography (PET) tracer or a single-photon emission computer tomography (SPECT) tracer comprising a triazolo[4,5-d]pyrimidine derivative of formula (I) for prognosis and/or diagnosis in-vivo of bacterial infection, or for in-vitro prognosis and/or diagnosis of bacterial infection.

In some embodiments, the tracer may be an imaging tracer. In some embodiments, the tracer may comprise the pharmaceutical composition.

The present invention will be illustrated in more detail in the following examples, which are not intended to limit the scope of the claimed invention in any way.

### EXAMPLE 1

### Preparation of ¹⁸F-Triafluocyl as Triazolo[4,5-d]Pyrimidine Derivative Comprising the Detectable Marker ¹⁸F

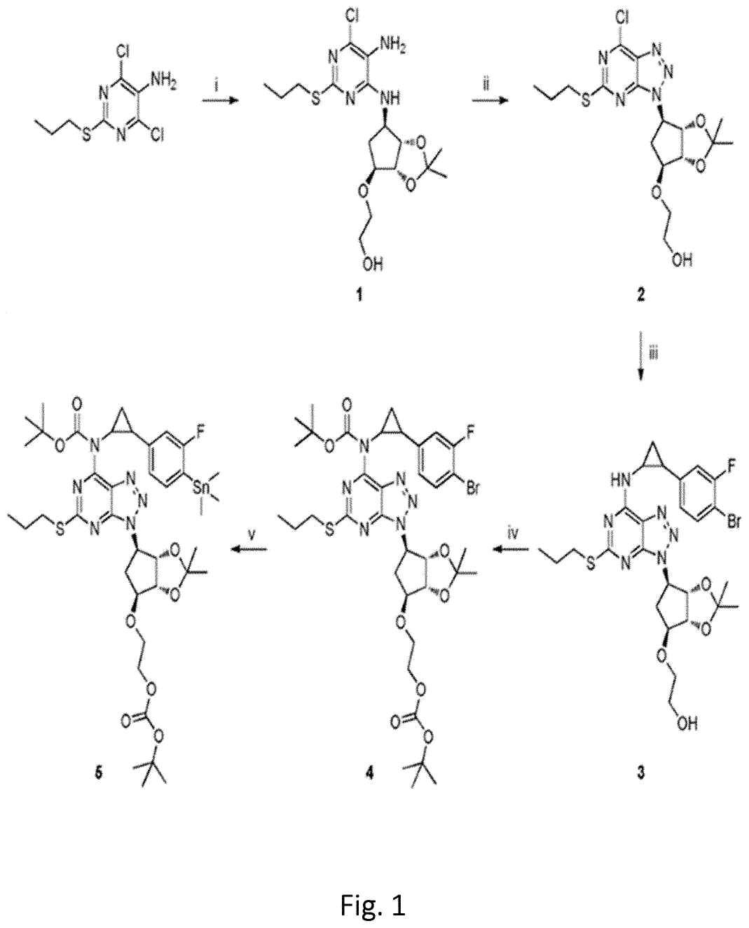

i) synthesis of a first labelling precursor illustrated in FIG. 1: tert-butyl (3-((3aS,4R,6S,6aR)-6-(2-((tert-butoxycarbonyl)oxy)ethoxy)-2,2-dimethyltetrahydro-3aH-cyclopenta[d][1,3]dioxol-4-yl)-5-(propylthio)-3H-[1,2,3]triazolo[4,5-d]pyrimidin-7-yl)((1R,2S)-2-(3-fluoro-4-(trimethylstannyl)phenyl)cyclopropyl)carbamate (5).

In a first step (i), 2-(((3aR,4S,6R,6aS)-6-((5-Amino-6-chloro-2-(propylthio)pyrimidin-4-yl)amino)-2,2-dimethyltetrahydro-3aH-cyclopenta[d][1,3]dioxol-4-yl)oxy)etanol (1) is obtained by reaction of 2-(((3aR,4S,6R,6aS)-6-amino-2,2-dimethyltetrahydro-3aH-cyclopenta[d][1,3]dioxol-4-yl)oxy)ethanol with 4,6-dichloro-2-(propylthio)pyrimidin-5-amine and is carried out in acetonitrile in a sealed vessel at 110° C.

The next step (ii) consists in a ring closure reaction of the intermediate 1 by means of a diazotization reaction with sodium nitrite in acetic acid at a temperature from 5° C. to 20° C. (step ii) leading to 2-(((3aR,4S,6R,6aS)-6-(7-chloro-5-(propylthio)-3H-[1,2,3]triazolo[4,5-d]pyrimidin-3-yl)-2,2-dimethyltetrahydro-3aH-cyclopenta[d][1,3]dioxol-4-yl)oxy)etanol (2).

In step iii, 2-(((3aR,4S,6R,6aS)-6-(7-(((1R,2S)-2-(4-bromo-3-fluorophenyl)cyclopropyl)amino)-5-(propylthio)-3H-[1,2,3]triazolo[4,5-d]pyrimidin-3-yl)-2,2-dimethyltetrahydro-3aH-cyclopenta[d][1,3]dioxol-4-yl)oxy)ethanol (3) is obtained by nucleophilic substitution of the chlorine atom of intermediate 2 by 2-(4-bromo-3-fluorophenyl)cyclopropanamine hydrochloride at a temperature of for example 20° C.

The sensitive amino and hydroxyl functions of 3 are then protected by a tert-butoxycarbonyl group (step iv) after reaction of 3 with di-tert-butyl dicarbonate in tetrahydrofuran at a temperature of for example 20° C. to give tert-butyl ((1R,2S)-2-(4-bromo-3-fluorophenyl)cyclopropyl)(3-((3aS,4R,6S,6aR)-6-(2-((tert-butoxycarbonyl)oxy)ethoxy)-2,2-dimethyltetrahydro-3aH-cyclopenta[d][1,3]dioxol-4-yl)-5-(propylthio)-3H-[1,2,3]triazolo[4,5-d]pyrimidin-7-yl)carbamate (4).

The labelling position is activated in a next step (step v) by nucleophilic substitution of the bromine atom of (4) by a trimethylstannyl group using hexamethyldistannane in the presence of tetrakis(triphenylphosphine)palladium(0) as a catalyst to give tert-butyl (3-((3aS,4R,6S,6aR)-6-(2-((tert-butoxycarbonyl)oxy)ethoxy)-2,2-dimethyltetrahydro-3aH-cyclopenta[d][1,3]dioxol-4-yl)-5-(propylthio)-3H-[1,2,3]triazolo[4,5-d]pyrimidin-7-yl)((1R,2S)-2-(3-fluoro-4-(trimethylstannyl)phenyl)cyclopropyl)carbamate (5).

The intermediate products (1) to (5) illustrated in FIG. 1 are hereafter called Intermediate (1) to intermediate (5)

Intermediate (1) is obtained as follows: a mixture of 4,6-dichloro-2-(propylthio)pyrimidin-5-amine (1.0 g, 4.2 mmol), 2-(((3aR,4S,6R,6aS)-6-amino-2,2-dimethyltetrahydro-3aH-cyclopenta[d][1,3]dioxol-4-yl)oxy)ethanol (1.17 g, 5.4 mmol) and triethylamine (0.6 mL, 4.2 mmol) in acetonitrile (10 mL) is introduced in a sealed vessel and heated overnight at 110° C. After evaporation of the solvent, the residue is purified by silica gel column chromatography.

Yield: 77%.

Melting point: 112-114° C.

¹H NMR (CDCl₃) δ 1.03 (t, J=7.4 Hz, 3H, SCH₂CH₂CH₃), 1.26 (s, 3H, CH₃), 1.43 (s, 3H, CH₃), 1.75 (m, 2H, SCH₂CH₂CH₃), 1.92 (d, J=14.5 Hz, 1H, 5′-Ha), 2.28 (ddd, J=14.5 Hz/5.9 Hz/4.4 Hz, 1H, 5′-Hb), 2.59 (bs, 1H, OH), 2.99 (ddd, J=13.4 Hz/8.2 Hz/6.4 Hz, 1H, SCHa), 3.14 (ddd, J=13.5 Hz/8.2 Hz/6.4 Hz, 1H, SCHb), 3.38 (bs, 2H, NH₂), 3.60 (ddd, J=9.9 Hz/6.1 Hz/2.6 Hz, 1H, OCHa), 3.70 (ddd, J=9.9 Hz/5.8 Hz/2.5 Hz, 1H, OCHb), 3.79 (m, 2H, OCH₂CH₂OH), 3.97 (d, J=4.1 Hz, 1H, 4′-H), 4.53 (dd, J=5.4 Hz/1.2 Hz, 1H, 3a′-H), 4.59 (m, 1H, 6′-H), 4.61 (dd, J=5.5 Hz/1.8 Hz, 1H, 6a′-H), 6.17 (d, J=8.4 Hz, 1H, NH). ¹³C NMR (CDCl₃) δ 13.5, 23.2, 23.8, 26.2, 32.5, 33.2, 56.8, 61.9, 70.4, 82.8, 84.5, 85.3, 110.3, 116.9, 144.5, 154.4, 162.0.

Intermediate (2) is obtained as follows: to a solution of (1) (1.0 g, 2.4 mmol) in acetic acid (10 mL) cooled on an ice bath is added NaNO₂ (225 mg, 3.2 mmol). The resulting mixture is allowed to reach room temperature within 1 hour and water (40 mL) is then added. The resulting mixture is extracted with ethyl acetate (3×50 mL) and the combined organic layers are dried over MgSO₄ and evaporated to give an oily residue.

Yield: 94%.

Melting point: oil.

¹H NMR (CDCl₃) δ 1.09 (t, J=7.4 Hz, 3H, SCH₂CH₂CH₃), 1.37 (s, 3H, CH₃), 1.55 (s, 3H, CH₃), 1.83 (h, J=7.4 Hz, 2H, SCH₂CH₂CH₃), 2.14 (t, J=6.0 Hz, 1H, OH), 2.54 (m, 1H, 5′-Ha), 2.70 (m, 1H, 5′-Hb), 3.21 (t, J=7.2 Hz, 2H, SCH₂CH₂CH₃), 3.49-3.65 (m, 4H, OCH₂CH₂OH), 4.05 (m, 1H, 4′-H), 4.88 (d, J=6.3 Hz, 1H, 3a′-H), 5.21 (td, J=7.4 Hz/6.4 Hz/2.5 Hz, 1H, 6′-H), 5.53 (dd, J=6.3 Hz/2.1 Hz, 1H, 6a′-H). ¹³C NMR (CDCl₃) δ 13.6, 22.3, 24.5, 26.8, 33.9, 35.9, 61.8, 63.4, 70.7, 82.9, 83.6, 84.0, 112.4, 132.2, 150.7, 153.4, 171.8.

Intermediate (3) is obtained by a mixing (2) (0.5 g, 1.16 mmol), 2-(4-bromo-3-fluorophenyl)cyclopropanamine hydrochloride (0.3 g, 1.16 mmol) with triethylamine (0.21 mL, 1.45 mmol) in acetonitrile (10 mL) and the resulting mixture is left to react at room temperature for 2 hours. After evaporation of the solvent, the residue is purified by silica gel column chromatography.

Yield: 98%.

Melting point: 122-124° C.

¹H NMR (DMSO-d₆) δ 0.82 (t, J=7.4 Hz, 2.4H, SCH₂CH₂CH₃ major), 0.99 (t, J=7.3 Hz, 0.6H, SCH₂CH₂CH₃ minor), 1.26 (s, 3H, CH₃), 1.42 (m, 1H, 3′-Ha), 1.48 (s, 3H, CH₃), 1.51 (m, 2H, SCH₂CH₂CH₃ major), 1.59 (dt, J=10.0 Hz/5.4 Hz, 1H, 3′-Hb), 1.70 (h, J=7.3 Hz, 0.4H, SCH₂CH₂CH₃ minor), 2.14 (ddd, J=9.6 Hz/6.4 Hz/3.3 Hz, 0.8H, 2′-H major), 2.24 (m, 0.2H, 2′-H minor), 2.52 (m, 1H, 5′″-Ha), 2.65 (m, 1H, 5′″-Hb), 2.87 (m, 1.6H, SCH₂CH₂CH₃ major), 3.08 (m, 0.4H, SCH₂CH₂CH₃ minor), 3.19 (dd, J=7.6 Hz/4.2 Hz, 0.8H, 1′-H major), 3.39-3.51 (m, 4H, OCH₂CH₂OH), 3.75 (m, 0.2H, 1′-H minor), 4.00 (m, 1H, 4′″-H), 4.56 (t, J=5.2 Hz, 1H, OH), 4.64 (m, 0.2H, 3a′″-H minor), 4.67 (dt, J=7.0 Hz/3.3 Hz, 0.8H, 3a′″-H major), 5.01 (m, 1H, 6′″-H), 5.16 (m, 0.2H, 6a′″-H minor), 5.20 (dt, J=7.3 Hz/4.8 Hz, 0.8H, 6a′″-H major), 6.97 (d, J=7.1 Hz, 0.2H, 6″-H minor), 7.03 (dd, J=8.3 Hz/1.9 Hz, 0.8H, 6″-H major), 7.18 (d, J=10.0 Hz, 0.2H, 2″-H minor), 7.24 (dd, J=10.4 Hz/1.9 Hz, 0.8H, 2″-H major), 7.59 (t, J=7.9 Hz, 1H, 5″-H), 9.01 (d, J=4.7 Hz, 0.2H, NH minor), 9.41 (dd, J=3.9 Hz/1.2 Hz, 0.8H, NH major). ¹³C NMR (DMSO-d₆) δ 13.0, 15.3, 22.3, 24.3, 24.8, 26.9, 32.4, 34.5, 35.4, 60.0, 61.4, 70.7, 81.9, 83.7, 104.5, 112.5, 114.1, 123.2, 123.9, 132.9, 144.3, 149.1, 153.9, 157.3, 159.3, 169.5.

Intermediate (4) is obtained by mixing (3) (0.62 g, 1.0 mmol), di-tert-butyl dicarbonate (1 g, 4.6 mmol), with 4-(dimethylamino)pyridine (30 mg, cat.) in tetrahydrofuran (10 mL) and the resulting mixture is left to react overnight at room temperature. After evaporation of the solvent, the residue is purified by silica gel column chromatography.

Yield: 56%

Melting point: 156-158° C.

¹H NMR (DMSO-d₆) δ 0.99 (td, J=7.3 Hz/2.6 Hz, 3H, SCH₂CH₂CH₃), 1.28 (s, 3H, CH₃), 1.35 (m, 1H, 3′-Ha), 1.39 (s, 9H, C(CH₃)₃), 1.40 (s, 9H, C(CH₃)₃), 1.50 (s, 3H, CH₃), 1.55 (qd, J=7.2 Hz/2.2 Hz, 1H, 3′-Hb), 1.70 (m, 2H, SCH₂CH₂CH₃), 2.26 (m, 1H, 2′-H), 2.61 (m, 1H, 5′″-Ha), 2.73 (m, 1H, 5′″-Hb), 3.05 (m, 2H, SCH₂CH₂CH₃), 3.27 (m, 1H, 1′-H), 3.62 (m, 2H, OCH₂CH₂OC(CH₃)₃), 4.04 (m, 3H, OCH₂CH₂OC(CH₃)₃/4′″-H), 4.71 (dd, J=7.2 Hz/3.0 Hz, 1H, 3a′″-H), 5.15 (m, 1H, 6′″-H), 5.27 (m, 1H, 6a′″-H), 7.02 (dd, J=8.3 Hz/1.9 Hz, 1H, 6″-H), 7.24 (dt, J=10.4 Hz/1.8 Hz, 1H, 2″-H), 7.59 (t, J=7.9 Hz, 1H, 5″-H). ¹³C NMR (DMSO-d₆) δ 13.3, 18.1, 22.1, 24.7, 26.0, 26.8, 27.3, 27.4, 32.6, 35.1, 39.0, 61.8, 65.5, 66.7, 81.4, 82.0, 82.1, 82.8, 83.6, 104.9, 112.5, 114.7, 124.3, 127.8, 132.9, 143.4, 150.6, 152.3, 152.9, 154.3, 157.2, 159.1, 169.1.

Precursor 1: tert-butyl (3-((3aS,4R,6S,6aR)-6-(2-((tert-butoxycarbonyl)oxy)ethoxy)-2,2-dimethyltetrahydro-3aH-cyclopenta[d][1,3]dioxol-4-yl)-5-(propylthio)-3H-[1,2,3]triazolo[4,5-d]pyrimidin-7-yl)((1R,2S)-2-(3-fluoro-4-(trimethylstannyl)phenyl)cyclopropyl)carbamate (5), is obtained by mixing (4) (0.41 g, 0.5 mmol), hexamethyldistannane (0.49 g, 1.5 mmol), with tetrakis(triphenylphosphine)palladium(0) (10 mg, cat.) in dry toluene (5 mL) and introduced in a sealed vessel and heated overnight at 100° C. under nitrogen. After cooling, ethyl acetate (50 mL) is added and the insoluble is filtered off. The filtrate is evaporated to dryness and the resulting residue is purified by silica gel column chromatography.

Yield: 65%

Melting point : 60-62° C.

¹H NMR (DMSO-d₆) δ 0.30 (s, 9H, Sn(CH₃)₃), 0.96 (td, J=7.3 Hz/2.9 Hz, 3H, SCH₂CH₂CH₃), 1.28 (s, 3H, CH₃), 1.32 (m, 1H, 3′-Ha), 1.39 (s, 9H, C(CH₃)₃), 1.40 (s, 9H, C(CH₃)₃), 1.50 (m, 4H, CH₃/3′-Hb), 1.67 (m, 2H, SCH₂CH₂CH₃), 2.23 (m, 1H, 2′-H), 2.58 (m, 1H, 5′″-Ha), 2.72 (m, 1H, 5′″-Hb), 3.03 (m, 2H, SCH₂CH₂CH₃), 3.24 (m, 1H, 1′-H), 3.61 (m, 2H, OCH₂CH₂OC(CH₃)₃), 4.03 (m, 3H, OCH₂CH₂OC(CH₃)₃/4′″-H), 4.71 (d, J=7.1 Hz, 1H, 3a′″-H), 5.15 (t, J=9.9 Hz, 1H, 6′″-H), 5.27 (dt, J=7.9 Hz/4.4 Hz, 1H, 6a′″-H), 6.95 (m, 1H, 2″-H), 7.02 (d, J=7.4 Hz, 1H, 6″-H), 7.59 (m, 1H, 5″-H). ¹³C NMR (DMSO-d₆) δ −8.9, 13.3, 17.9, 22.1, 24.7, 26.2, 26.8, 27.3, 27.4, 32.6, 35.1, 39.0, 61.8, 65.5, 66.7, 81.4, 82.0, 82.1, 82.7, 83.6, 112.1, 112.5, 122.7, 123.6, 127.9, 136.4, 144.2, 150.6, 152.4, 152.9, 154.4, 166.1, 167.9, 169.1.

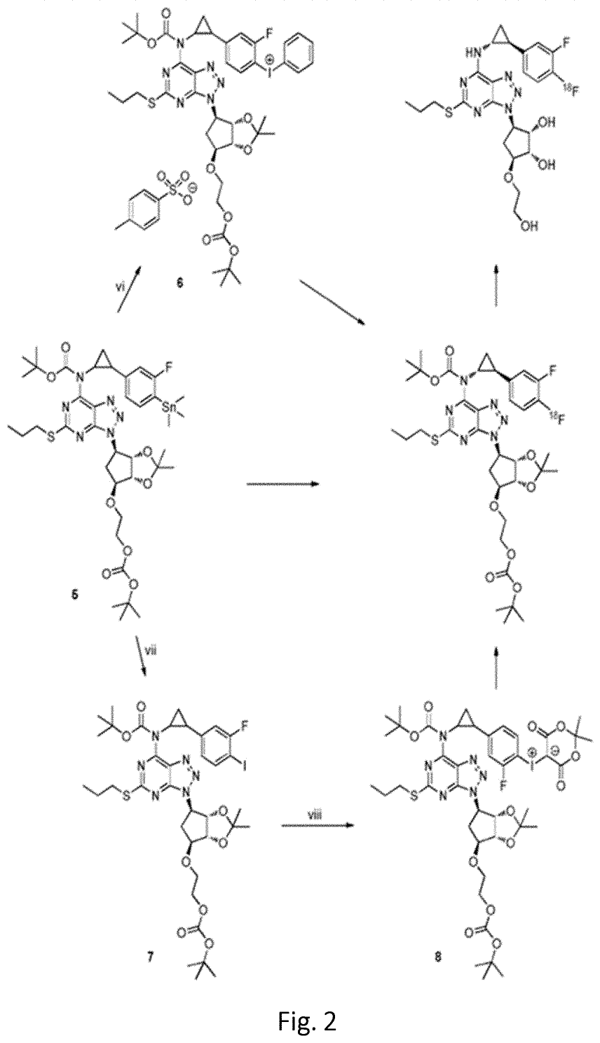

ii) Synthesis of Further Labelling Precursors as Illustrated in FIG. 2

Other precursors can be synthesized from (5) using hydroxy(tosyloxy)iodobenzene (step vi) to give the corresponding iodonium tosylate (6), or using iodine (step vii) to give (7) followed by addition of Meldrum's acid (step viii) to give the iodonium ylide (8).

Labelling precursor (6) is obtained by adding hydroxy(tosyloxy)iodobenzene (0.13 g, 0.33 mmol) to a solution of (5) (0.27 g, 0.3 mmol) in dichloromethane (5 mL) at 0° C. The resulting mixture is left to react at room temperature for 1 hour. After evaporation of the solvent, the residue is purified by silica gel column chromatography.

Yield: 72%.

Melting point: 92-95° C.

¹H NMR (DMSO-d₆) δ 0.96 (t, J=7.0 Hz, 3H, SCH₂CH₂CH₃), 1.27 (s, 3H, CH₃), 1.37 (s, 9H, C(CH₃)₃), 1.39 (s, 9H, C(CH₃)₃), 1.43 (m, 1H, 3′-Ha), 1.50 (s, 3H, CH₃), 1.63 (m, 3H, SCH₂CH₂CH₃/3′-Hb), 2.29 (s, 3H, CH_{3 tos}), 2.32 (m, 1H, 2′-H), 2.57 (m, 1H, 5′″-Ha), 2.72 (m, 1H, 5′″-Hb), 2.83-3.07 (m, 2H, SCH₂CH₂CH₃), 3.30 (m, 1H, 1′-H), 3.62 (m, 2H, OCH₂CH₂OC(CH₃)₃), 4.04 (m, 3H, OCH₂CH₂OC(CH₃)₃/4′″-H), 4.70 (dd, J=7.2 Hz/2.9 Hz, 1H, 3a′″-H), 5.14 (m, 1H, 6′″-H), 5.25 (dd, J=7.0 Hz/4.6 Hz, 1H, 6a′″-H), 7.11 (d, J=7.8 Hz, 2H, 3-Hₜₒₛ/5-Hₜₒₛ), 7.20 (dd, J=8.4 Hz/1.7 Hz, 1H, 6″-H), 7.41 (dd, J=10.0 Hz/1.6 Hz, 1H, 2″-H), 7.47 (d, J=8.1 Hz, 2H, 2-Hₜₒₛ/6-Hₜₒₛ), 7.54 (t, J=7.8 Hz, 2H, 3″″-H/5″″-H), 7.68 (t, J=7.4 Hz, 1H, 4″″-H), 8.21 (d, J=7.6 Hz, 2H, 2″″-H/6″″-H), 8.29 (dd, J=8.2 Hz/6.6 Hz, 1H, 5″-H). ¹³C NMR (DMSO-d₆) δ 13.2, 19.0, 20.8, 22.1, 24.7, 26.5, 26.8, 27.3, 27.4, 32.5, 35.2, 39.9, 61.8, 65.5, 66.7, 81.4, 82.0, 82.9, 83.6, 100.3, 112.5, 114.5, 117.0, 125.5, 127.8, 128.0, 131.9, 132.2, 135.0, 136.5, 137.5, 145.9, 150.6, 152.2, 152.9, 154.1, 158.3, 160.3, 169.0.

Labelling precursor tert-butyl (3-((3aS,4R,6S,6aR)-6-(2-((tert-butoxycarbonyl)oxy)ethoxy)-2,2-dimethyltetrahydro-3aH-cyclopenta[d][1,3]dioxol-4-yl)-5-(propylthio)-3H-[1,2,3]triazolo[4,5-d]pyrimidin-7-yl)((1R,2S)-2-(3-fluoro-4-iodophenyl)cyclopropyl)carbamate (7) is obtained by adding Iodine (0.15 g, 0.6 mmol) to a solution of (5) (0.27 g, 0.3 mmol) in dichloromethane (5 mL). The resulting mixture is left to react at room temperature for 1 hour. After evaporation of the solvent, the residue is purified by silica gel column chromatography.

Yield: 61%.

Melting point: 151-153° C.

¹H NMR (DMSO-d₆) δ 0.98 (t, J=7.3 Hz, 3H, SCH₂CH₂CH₃), 1.28 (s, 3H, CH₃), 1.34 (m, 1H, 3′-Ha), 1.39 (s, 9H, C(CH₃)₃), 1.40 (s, 9H, C(CH₃)₃), 1.50 (s, 3H, CH₃), 1.53 (q, J=6.9 Hz, 1H, 3′-Hb), 1.70 (h, J=7.3 Hz, 2H, SCH₂CH₂CH₃), 2.24 (m, 1H, 2′-H), 2.59 (m, 1H, 5′″-Ha), 2.72 (m, 1H, 5′″-Hb), 3.05 (m, 2H, SCH₂CH₂CH₃), 3.26 (m, 1H, 1′-H), 3.56-3.68 (m, 2H, OCH₂CH₂OC(CH₃)₃), 4.03 (m, 3H, OCH₂CH₂₀C(CH₃)₃/4′″-H), 4.70 (dd, J=7.2 Hz/3.0 Hz, 1H, 3a′″-H), 5.15 (m, 1H, 6′″-H), 5.26 (m, 1H, 6a′″-H), 6.87 (dd, J=8.2 Hz/1.8 Hz, 1H, 6″-H), 7.13 (dt, J=9.7 Hz/1.8 Hz, 1H, 2″-H), 7.71 (dd, J=7.9 Hz/7.1 Hz, 1H, 5″-H). ¹³C NMR (DMSO-d₆) δ 13.3, 18.1, 22.1, 24.7, 26.1, 26.8, 27.3, 27.4, 31.0, 32.6, 35.1, 39.1, 61.9, 65.5, 66.7, 78.3, 78.5, 81.4, 81.9, 82.0, 82.7, 83.6, 112.5, 113.7, 113.9, 124.7, 127.9, 138.6, 144.0, 150.6, 152.3, 152.9, 154.3, 160.2, 162.1, 169.1.

iii) Conversion of Labelling Precursors 6 and 8 into ¹⁸F-Triafluocyl.

the iodonium group of both labelling precursor 6 and 8 are converted into ¹⁸F-Triafluocyl according to described processes in the literature:

Regarding precursor 6 in Copper-Mediated Radiofluorination of Arylstannanes with [18F]KF; by Makaravage, Katarina J.; Brooks, Allen F.; Mossine, Andrew V.; Sanford, Melanie S.; Scott, Peter J. H. (from Organic Letters (2016), 18(20), 5440-5443).

Regarding precursor 8 in Spirocyclic hypervalent iodine(III)-mediated radiofluorination of non-activated and hindered aromatics; by Rotstein, Benjamin H.; Stephenson, Nickeisha A.; Vasdev, Neil; Liang, Steven H. (From Nature Communications (2014), 5, 4365).

### EXAMPLE 2

### Comparison of In Vitro ¹⁸F-Triafluocyl and ¹⁸F-FDG Uptake Assay

In order to assess the selective uptake of ¹⁸F-Triafluocyl into bacteria and its utility for the specific diagnosis of bacterial infections, we performed an in vitro assay in which we compared the uptake of ¹⁸F-triafluocyl and ¹⁸F-FDG into bacteria.

For this purpose, S. epidermidis bacteria were incubated with ¹⁸F-FDG or ¹⁸F-Triafluocyl, and the relative radioactivity associated with the bacterial cells was determined as follows.

S. epidermidis bacteria were grown overnight in tryptic soy broth (TSB) at 37° C., with shaking at 250 rpm. The overnight culture was diluted to OD₆₀₀ 0.1 and incubated until mid-exponential phase was reached. 1×10⁸ CFU were resuspended in 1 ml of a cell culture medium RPMI 1640 provided by Sigma-Aldrich (R7638).

Bacteria and control without bacteria were incubated with 2 MBq ¹⁸F-FDG or 2 MBq ¹⁸F-Triafluocyl prepared as described in Example 1 for 1 h at 37° C. Bacteria were harvested by centrifugation (600×g, 5 min) and washed three times by successive centrifugations. After washing, the cells were transferred into scintillation vials. The supernatants were also collected in scintillation vials. Bacteria and supernatants were counted by gamma counter (2470 Wizard²™ (Perkin Elmer))

Results were obtained as counts per min. Results were normalised for controls (no bacteria) and by calculating the percentage of activity in the cells-containing scintillation vials compared to the total counts (cells and supernatants combined).

After incubation with the bacteria, we found that the relative activity of ¹⁸F-triafluocyl associated with the bacterial cells was 1.5 to 2-fold higher than the activity of ¹⁸F-FDG.

### EXAMPLE 3

### Use of In-Vitro ¹⁸F-Triafluocyl for Prognosis and/or Diagnosis of Bacterial Infection From a Blood Sample Obtained From a Host Mammal (Human)

Since it is well-known in the art that Triafluocyl (also called Ticagrelor), binds the platelet P2Y12 receptor reversibly, and platelets accumulate at sites of bacterial infection (as described in Hamzeh-Cognasse H, Damien P, Chabert A, Pozzetto B, Cognasse F, Garraud O. Platelets and infections—complex interactions with bacteria. Front Immunol. 2015; 6:82. Published 2015 Feb. 26. doi:10.3389/fimmu.2015.00082), we also compared radiotracer uptake into human platelets in the presence or in the absence of bacteria.

Preparation of human washed platelets: a Blood sample was collected from healthy volunteers on Acid Citrate Dextrose (ACD: 93 mM Na₃-citrate, 7 mM citric acid, 14 mM dextrose, pH 6.0) containing 1 U/ml apyrase in a volume ratio of ACD to blood of 1:6. Blood was centrifuged for 5 s at 800×g followed by 5 min at 100×g to obtain platelet rich plasma (PRP). PRP was diluted 3-fold in ACD containing 1 U/ml apyrase ((Apyrase from potatoes, Grade I (A6132 Sigma-Aldrich)) and centrifuged at 1000×g to obtain a platelet pellet. The platelet pellet was resuspended at a concentration of 3×10⁸ ml⁻¹ in Tyrode's buffer (137 mM NaCl, 12 mM NaHCO₃, 2 mM KCl, 0.34 mM Na₂HPO₄, 1 mM MgCl₂, 5.5 mM glucose, 5 mM Hepes, 0.35% Bovine Serum Albumine from Sigma Aldrich A3294) and Hepes refers to 4-(2-Hydroxyethyl)piperazine-1-ethanesulfonic acid, N-(2-Hydroxyethyl)piperazine-N′-(2-ethanesulfonic acid) (H4034, Sigma-Aldrich).

Bacteria, platelets and controls without bacteria were incubated with 2 MBq ¹⁸F-FDG or 2 MBq ¹⁸F-Triafluocyl for 1 h at 37° C. Bacteria and platelets were harvested by centrifugation (1000×g, 10 min) and washed three times by successive centrifugations (1000×g for 10 min). Supernatants were collected in scintillation vials. After washing, the cells were transferred into scintillation vials. Cells and supernatants were counted by gamma counter (2470 Wizard²™ (Perkin Elmer))

Results were obtained as counts per min. Results were normalised for controls (no cells) and by calculating the percentage of activity in the cell containing scintillation vials compared with the total counts (cells and supernatants combined).

Results confirm ¹⁸F-triafluocyl uptake into platelets, while ¹⁸F-FDG was not transported into these cells. Moreover, after incubation with platelet suspensions containing bacteria, we found that the relative activity of ¹⁸F-triafluocyl associated with the cell mixture was about 10-fold higher than the activity of ¹⁸F-FDG.

### EXAMPLE 4

### Selective Use of ¹⁸F-Triafluocyl for Prognosis and/or Diagnosis of Bacterial Infection Over Mammalian Cells Obtained From a Human Host

S. epidermidis bacteria were grown overnight in tryptic soy broth (TSB) at 37° C., with shaking at 250 rpm. The overnight culture was diluted to OD₆₀₀ 0.1 and incubated until mid-exponential phase was reached. 1×10⁸ CFU were resuspended in 1 ml RPMI 1640.

THP1 (ATCC® TIB-202™) and HL60 (ATCC® CCL-240™) cell lines were grown in RPMI 1640 tissue culture medium supplemented with L-glutamine, 10% foetal calf serum and 1% penicillin/streptomycin at 37° C. and 5% CO₂. HT29 cell line (ATCC® HTB-38) was grown in McCoy's 5A medium and 10% foetal bovine serum at 37° C. and 5% CO₂.

Non-adherent cell lines (HL60, THP1; 1×10⁶ cells ml⁻¹) were harvested, washed and resuspended in 1 ml tissue culture medium. The adherent cells HT29 were maintained in 6-well plates at 80% confluence.

Bacteria, cell lines and controls without cells were incubated with 2 MBq ¹⁸F-FDG or 2 MBq ¹⁸F-ticagrelor for 1 h at 37° C. Bacteria and non-adherent cells were harvested by centrifugation (600×g, 5 min) and washed three times by successive centrifugations. Cell supernatants were collected in scintillation vials. After washing, the cells were transferred into scintillation vials. Adherent cells were washed three times with fresh medium (McCoy's 5A medium plus 10% foetal bovine serum). Supernatants were collected into scintillation vials. Adherent cells were detached by trypsin treatment and placed into scintillation vials. The scintillation vials for cells and supernatants were counted by gamma counter. Results were obtained as counts per min. Results were normalised for controls (no cells) and by calculating the percentage of activity in the cell containing scintillation vials compared with the total counts (cells and supernatant combined).

Results confirm ¹⁸F-Triafluocyl uptake into bacteria while no uptake was observed into any of the mammalian cells, leukocytic (THP1 and HL-60) or tumor (HT29) cells. In contrast, we observed an uptake of ¹⁸F-FDG into the three lines of mammalian cells. ¹⁸F-Triafluocyl can thus be used for the specific in vitro detection of bacterial infection in a sample of human origin.

### EXAMPLE 5

### Protocol of In-Vivo Prognosis and/or Diagnosis

A protocol of test for in-vivo prognosis and/or diagnosis of bacterial infection in a patient has been established and adapted to the use of ¹⁸F-Triafluocyl as radiotracer.

The protocol hereafter has been developed for PET-CT imaging but may easily be extrapolated by the man skilled in the art, to other imaging technics such as SPECT.

The protocol is based on the same image acquisition for each patient:

After a minimum of 6 h fasting, 3.7 MBq ¹⁸F-Triafluocyl/Kg body weight (mean activity/patient: 277 MBq, range: 202-394 MBq) is injected through a peripheral vein catheter. The patient is placed into a quiet room and instructed not to move. Approximately 1 h (mean: 69 min, range: 54-100 min) after injection of 18F-Triafluocyl, static whole-body examination is performed with a PET-CT scanner. Volumetric low-dose axial CT images are acquired. Then, emission raw data images are recorded at each bed and reconstructed as overlapping coronal slices after CT attenuation model-based scatter correction (convolution subtraction) and normalization correction.

The protocol can be applied to patients suffering from a bacterial infection allowing a deep-seated location in all tissues of the patient's body such as muscle, epithelial, connective and nervous, whereas patients suffering from cancer or sterile inflammation are less detectable by the present protocol.

### EXAMPLE 6

### Best Mode of Preparation of ¹⁸F-Triafluocyl

In a first step (i), 2-(((3aR,4S,6R,6aS)-6-((5-Amino-6-chloro-2-(propylthio)pyrimidin-4-yl)amino)-2,2-dimethyltetra hydro-3aH-cyclopenta[d][1,3]dioxol-4-yl)oxy)etanol (1) is obtained by reaction of 2-(((3aR,4S,6R,6aS)-6-amino-2,2-dimethyltetrahydro-3aH-cyclopenta[d][1,3]dioxol-4-yl)oxy)ethanol with 4,6-dichloro-2-(propylthio)pyrimidin-5-amine in acetonitrile at a temperature of for example 110° C.

This step is followed by a ring closure reaction of intermediate 1 by means of sodium nitrite carried out in acetic acid at a temperature from 5° C. to 20° C. (step ii) to obtain 2-(((3aR,4S,6R,6aS)-6-(7-chloro-5-(propylthio)-3H-[1,2,3]triazolo[4,5-d]pyrimidin-3-yl)-2,2-dimethyltetrahydro-3aH-cyclopenta[d][1,3]dioxol-4-yl)oxy)etanol (2).

In step ix, 2-(((3aR,4S,6R,6aS)-6-(7-((2-(4-bromo-3-fluorophenyl)cyclopropyl)amino)-5-(propylthio)-3H-[1,2,3]triazolo[4,5-d]pyrimidin-3-yl)-2,2-dimethyltetrahydro-3aH-cyclopenta[d][1,3]dioxol-4-yl)oxy)ethanol (9) is obtained by nucleophilic substitution of the chlorine atom of intermediate 2 by 2-(4-bromo-3-fluorophenyl)cyclopropanamine hydrochloride in the presence of a base such as triethylamine at a temperature of 20° C.

The sensitive functions of 9 are then protected by the tert-butoxycarbonyl group (step x) after reaction of 9 with di-tert-butyl dicarbonate in tetrahydrofurane at a temperature of for example 20° C. to give tert-butyl (2-(4-bromo-3-fluorophenyl)cyclopropyl)(3-((3aS,4R,6S,6aR)-6-(2-((tert-butoxycarbonyl)oxy)ethoxy)-2,2-dimethyltetrahydro-3aH-cyclopenta[d][1,3]dioxol-4-yl)-5-(propylthio)-3H-[1,2,3]triazolo[4,5-d]pyrimidin-7-yl)carbamate (10).

The labelling position is activated in a next step (step xi) by nucleophilic substitution of the bromine atom of (10) by a trimethylstannyl group using hexamethyldistannane in the presence of tetrakis(triphenylphosphine)palladium(0) as a catalyst to give tert-butyl (3-((3aS,4R,6S,6aR)-6-(2-((tert-butoxycarbonyl)oxy)ethoxy)-2,2-dimethyltetrahydro-3aH-cyclopenta[d][1,3]dioxol-4-yl)-5-(propylthio)-3H-[1,2,3]triazolo[4,5-d]pyrimidin-7-yl)(2-(3-fluoro-4-(trimethylstannyl)phenyl)cyclopropyl)carbamate (11), followed by a reaction with hydroxy(tosyloxy)iodo-4-methoxybenzene (12) (step xii) to give the corresponding iodonium tosylate (13).

1. Preparation of Labelling Precursor:

### (Example 1:) synthesis of (4-(2-((tert-butoxycarbonyl)(3-((3aS,4R,6S,6aR)-6-(2-((tert-butoxycarbonyl)oxy)ethoxy)-2,2-dimethyltetrahydro-3aH-cyclopenta[d][1,3]dioxol-4-yl)-5-(propylthio)-3H-[1,2,3]triazolo[4,5-d]pyrimidin-7-yl)amino)cyclopropyl)-2-fluorophenyl)(4-methoxyphenyl)iodonium tosylate (13)

### 2-(((3aR,4S,6R,6aS)-6-((5-Amino-6-chloro-2-(propylthio)pyrimidin-4-yl)amino)-2,2-dimethyltetrahydro-3aH-cyclopenta[d][1,3]dioxol-4-yl)oxy)etanol (1)

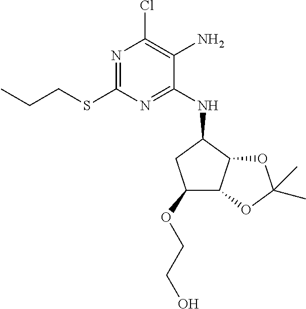 [SMILES: CCCSc1nc(Cl)c(N)c(N[C@@H]2C[C@H](OCCO)[C@H]3OC(C)(C)O[C@H]32)n1]

The mixture of 4,6-dichloro-2-(propylthio)pyrimidin-5-amine (1.0 g, 4.2 mmol), 2-(((3aR,4S,6R,6aS)-6-amino-2,2-dimethyltetrahydro-3aH-cyclopenta[d][1,3]dioxol-4-yl)oxy)ethanol (1.17 g, 5.4 mmol) and triethylamine (0.6 mL, 4.2 mmol) in acetonitrile (10 mL) was introduced in a sealed vessel and heated overnight at 110° C. After evaporation of the solvent, the residue was purified by silica gel column chromatography.

Yield: 77%.

Melting point: 112-114° C.

¹H NMR (CDCl₃) δ 1.03 (t, J=7.4 Hz, 3H, SCH₂CH₂CH₃), 1.26 (s, 3H, CH₃), 1.43 (s, 3H, CH₃), 1.75 (m, 2H, SCH₂CH₂CH₃), 1.92 (d, J=14.5 Hz, 1H, 5′-Ha), 2.28 (ddd, J=14.5 Hz/5.9 Hz/4.4 Hz, 1H, 5′-Hb), 2.59 (bs, 1H, OH), 2.99 (ddd, J=13.4 Hz/8.2 Hz/6.4 Hz, 1H, SCHa), 3.14 (ddd, J=13.5 Hz/8.2 Hz/6.4 Hz, 1H, SCHb), 3.38 (bs, 2H, NH₂), 3.60 (ddd, J=9.9 Hz/6.1 Hz/2.6 Hz, 1H, OCHa), 3.70 (ddd, J=9.9 Hz/5.8 Hz/2.5 Hz, 1H, OCHb), 3.79 (m, 2H, OCH₂CH₂OH), 3.97 (d, J=4.1 Hz, 1H, 4′-H), 4.53 (dd, J=5.4 Hz/1.2 Hz, 1H, 3a′-H), 4.59 (m, 1H, 6′-H), 4.61 (dd, J=5.5 Hz/1.8 Hz, 1H, 6a′-H), 6.17 (d, J=8.4 Hz, 1H, NH). ¹³C NMR (CDCl₃) δ 13.5, 23.2, 23.8, 26.2, 32.5, 33.2, 56.8, 61.9, 70.4, 82.8, 84.5, 85.3, 110.3, 116.9, 144.5, 154.4, 162.0.

### 2-(((3aR,4S,6R,6aS)-6-(7-chloro-5-(propylthio)-3H-[1,2,3]triazolo[4,5-d]pyrimidin-3-yl)-2,2-dimethyltetrahydro-3aH-cyclopenta[d][1,3]dioxol-4-yl)oxy)etanol (2)

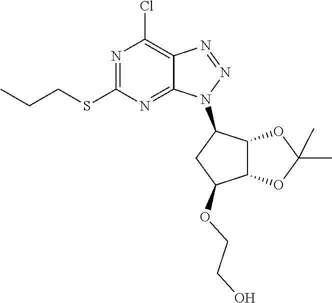 [SMILES: CCCSc1nc(Cl)c2nnn([C@@H]3C[C@H](OCCO)[C@H]4OC(C)(C)O[C@H]43)c2n1]

To a solution of (1) (1.0 g, 2.4 mmol) in acetic acid (10 mL) cooled on an ice bath was added NaNO₂ (225 mg, 3.2 mmol). The mixture was allowed to reach room temperature within 1 hour and water (40 mL) was then added. The resulting mixture was extracted with ethyl acetate (3×50 mL) and the combined organic layers were dried over MgSO₄ and evaporated to give an oily residue.

Yield: 94%.

Melting point: oil.

¹H NMR (CDCl₃) δ 1.09 (t, J=7.4 Hz, 3H, SCH₂CH₂CH₃), 1.37 (s, 3H, CH₃), 1.55 (s, 3H, CH₃), 1.83 (h, J=7.4 Hz, 2H, SCH₂CH₂CH₃), 2.14 (t, J=6.0 Hz, 1H, OH), 2.54 (m, 1H, 5′-Ha), 2.70 (m, 1H, 5′-Hb), 3.21 (t, J=7.2 Hz, 2H, SCH₂CH₂CH₃), 3.49-3.65 (m, 4H, OCH₂CH₂OH), 4.05 (m, 1H, 4′-H), 4.88 (d, J=6.3 Hz, 1H, 3a′-H), 5.21 (td, J=7.4 Hz/6.4 Hz/2.5 Hz, 1H, 6′-H), 5.53 (dd, J=6.3 Hz/2.1 Hz, 1H, 6a′-H). ¹³C NMR (CDCl₃) δ 13.6, 22.3, 24.5, 26.8, 33.9, 35.9, 61.8, 63.4, 70.7, 82.9, 83.6, 84.0, 112.4, 132.2, 150.7, 153.4, 171.8.

### 2-(((3aR,4S,6R,6aS)-6-(7-((2-(4-bromo-3-fluorophenyl)cyclopropyl)amino)-5-(propylthio)-3H-[1,2,3]triazolo[4,5-d]pyrimidin-3-yl)-2,2-dimethyltetrahydro-3aH-cyclopenta[d][1,3]dioxol-4-yl)oxy)ethanol (9)

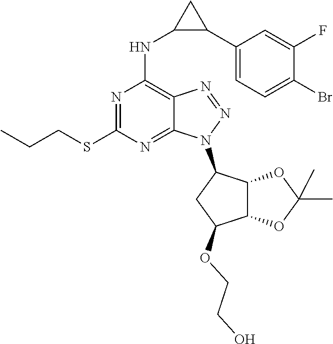 [SMILES: CCCSc1nc(NC2CC2c2ccc(Br)c(F)c2)c2nnn([C@@H]3C[C@H](OCCO)[C@H]4OC(C)(C)O[C@H]43)c2n1]

The mixture of (2) (0.5 g, 1.16 mmol), 2-(4-bromo-3-fluorophenyl)cyclopropanamine hydrochloride (0.3 g, 1.16 mmol) and triethylamine (0.21 mL, 1.45 mmol) in acetonitrile (10 mL) was left to react at room temperature for 2 hours. After evaporation of the solvent, the residue was purified by silica gel column chromatography.

Yield: 98%.

Melting point: 122-124° C.

¹H NMR (DMSO-d₆) δ 0.82 (t, J=7.4 Hz, 2.4H, SCH₂CH₂CH₃ major), 0.99 (t, J=7.3 Hz, 0.6H, SCH₂CH₂CH₃ minor), 1.26 (s, 3H, CH₃), 1.42 (m, 1H, 3′-Ha), 1.48 (s, 3H, CH₃), 1.51 (m, 2H, SCH₂CH₂CH₃ major), 1.59 (dt, J=10.0 Hz/5.4 Hz, 1H, 3′-Hb), 1.70 (h, J=7.3 Hz, 0.4H, SCH₂CH₂CH₃ minor), 2.14 (ddd, J=9.6 Hz/6.4 Hz/3.3 Hz, 0.8H, 2′-H major), 2.24 (m, 0.2H, 2′-H minor), 2.52 (m, 1H, 5′″-Ha), 2.65 (m, 1H, 5′″-Hb), 2.87 (m, 1.6H, SCH₂CH₂CH₃ major), 3.08 (m, 0.4H, SCH₂CH₂CH₃ minor), 3.19 (dd, J=7.6 Hz/4.2 Hz, 0.8H, 1′-H major), 3.39-3.51 (m, 4H, OCH₂CH₂OH), 3.75 (m, 0.2H, 1′-H minor), 4.00 (m, 1H, 4′″-H), 4.56 (t, J=5.2 Hz, 1H, OH), 4.64 (m, 0.2H, 3a′″-H minor), 4.67 (dt, J=7.0 Hz/3.3 Hz, 0.8H, 3a′″-H major), 5.01 (m, 1H, 6′″-H), 5.16 (m, 0.2H, 6a′″-H minor), 5.20 (dt, J=7.3 Hz/4.8 Hz, 0.8H, 6a′″-H major), 6.97 (d, J=7.1 Hz, 0.2H, 6″-H minor), 7.03 (dd, J=8.3 Hz/1.9 Hz, 0.8H, 6″-H major), 7.18 (d, J=10.0 Hz, 0.2H, 2″-H minor), 7.24 (dd, J=10.4 Hz/1.9 Hz, 0.8H, 2″-H major), 7.59 (t, J=7.9 Hz, 1H, 5″-H), 9.01 (d, J=4.7 Hz, 0.2H, NH minor), 9.41 (dd, J=3.9 Hz/1.2 Hz, 0.8H, NH major). ¹³C NMR (DMSO-d₆) δ 13.0, 15.3, 22.3, 24.3, 24.8, 26.9, 32.4, 34.5, 35.4, 60.0, 61.4, 70.7, 81.9, 83.7, 104.5, 112.5, 114.1, 123.2, 123.9, 132.9, 144.3, 149.1, 153.9, 157.3, 159.3, 169.5.

### tert-butyl (2-(4-bromo-3-fluorophenyl)cyclopropyl)(3-((3aS,4R,6S,6aR)-6-(2-((tert-butoxycarbonyl)oxy)ethoxy)-2,2-dimethyltetrahydro-3aH-cyclopenta[d][1,3]dioxol-4-yl)-5-(propylthio)-3H-[1,2,3]triazolo[4,5-d]pyrimidin-7-yl)carbamate (10)

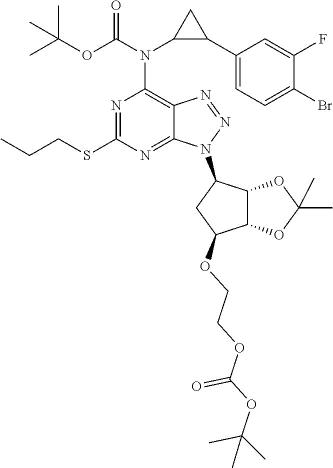 [SMILES: CCCSc1nc(N(C(=O)OC(C)(C)C)C2CC2c2ccc(Br)c(F)c2)c2nnn([C@@H]3C[C@H](OCCOC(=O)OC(C)(C)C)[C@H]4OC(C)(C)O[C@H]43)c2n1]

The mixture of (9) (0.62 g, 1.0 mmol), di-tert-butyl dicarbonate (1 g, 4.6 mmol), and 4-(dimethylamino)pyridine (30 mg, cat.) in tetrahydrofurane (10 mL) was left to react overnight at room temperature. After evaporation of the solvent, the residue was purified by silica gel column chromatography.

Yield: 56%

Melting point: 156-158.5° C.

¹H NMR (DMSO-d₆) δ 0.98 (td, J=7.3 Hz/2.6 Hz, 3H, SCH₂CH₂CH₃), 1.28 (s, 3H, CH₃), 1.35 (m, 1H, 3′-Ha), 1.39 (s, 9H, C(CH₃)₃), 1.40 (s, 9H, C(CH₃)₃), 1.50 (s, 3H, CH₃), 1.55 (qd, J=7.2 Hz/2.2 Hz, 1H, 3′-Hb), 1.70 (m, 2H, SCH₂CH₂CH₃), 2.26 (m, 1H, 2′-H), 2.61 (m, 1H, 5′″-Ha), 2.73 (m, 1H, 5′″-Hb), 3.05 (m, 2H, SCH₂CH₂CH₃), 3.27 (m, 1H, 1′-H), 3.62 (m, 2H, OCH₂CH₂OC(CH₃)₃), 4.04 (m, 3H, OCH₂CH₂OC(CH₃)₃/4′″-H), 4.71 (dd, J=7.2 Hz/3.0 Hz, 1H, 3a′″-H), 5.15 (m, 1H, 6′″-H), 5.27 (m, 1H, 6a′″-H), 7.02 (dd, J=8.3 Hz/1.9 Hz, 1H, 6″-H), 7.24 (dt, J=10.4 Hz/1.8 Hz, 1H, 2″-H), 7.59 (t, J=7.9 Hz, 1H, 5″-H). ¹³C NMR (DMSO-d₆) δ 13.3, 18.1, 22.1, 24.7, 26.0, 26.8, 27.3, 27.4, 32.6, 35.1, 39.0, 61.8, 65.5, 66.7, 81.4, 82.0, 82.1, 82.8, 83.6, 104.9, 112.5, 114.7, 124.3, 127.8, 132.9, 143.4, 150.6, 152.3, 152.9, 154.3, 157.2, 159.1, 169.1.

### tert-butyl (3-((3aS,4R,6S,6aR)-6-(2-((tert-butoxycarbonyl)oxy)ethoxy)-2,2-dimethyltetrahydro-3aH-cyclopenta[d][1,3]dioxol-4-yl)-5-(propylthio)-3H-[1,2,3]triazolo[4,5-d]pyrimidin-7-yl)(2-(3-fluoro-4-(trimethylstannyl)phenyl)cyclopropyl)carbamate (11)

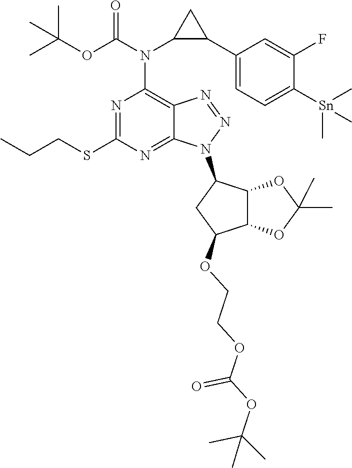 [SMILES: CCCSc1nc(N(C(=O)OC(C)(C)C)C2CC2c2cc[c]([Sn]([CH3])([CH3])[CH3])c(F)c2)c2nnn([C@@H]3C[C@H](OCCOC(=O)OC(C)(C)C)[C@H]4OC(C)(C)O[C@H]43)c2n1]

The mixture of (10) (0.41 g, 0.5 mmol), hexamethyldistannane (0.49 g, 1.5 mmol), and tetrakis(triphenylphosphine)palladium(0) (10 mg, cat.) in dry toluene (5 mL) was introduced in a sealed vessel and heated overnight at 100° C. under nitrogen. After cooling, ethyl acetate (50 mL) was added and the insoluble was filtered off. The filtrate was evaporated to dryness and the resulting residue was purified by silica gel column chromatography.

Yield: 65%

Melting point: 75-77° C.

¹H NMR (DMSO-d₆) δ 0.30 (s, 9H, Sn(CH₃)₃), 0.96 (td, J=7.3 Hz/2.9 Hz, 3H, SCH₂CH₂CH₃), 1.28 (s, 3H, CH₃), 1.32 (m, 1H, 3′-Ha), 1.39 (s, 9H, C(CH₃)₃), 1.40 (s, 9H, C(CH₃)₃), 1.50 (m, 4H, CH₃/3′-Hb), 1.67 (m, 2H, SCH₂CH₂CH₃), 2.23 (m, 1H, 2′-H), 2.58 (m, 1H, 5′″-Ha), 2.72 (m, 1H, 5′″-Hb), 3.03 (m, 2H, SCH₂CH₂CH₃), 3.24 (m, 1H, 1′-H), 3.61 (m, 2H, OCH₂CH₂OC(CH₃)₃), 4.03 (m, 3H, OCH₂CH₂OC(CH₃)₃/4′″-H), 4.71 (d, J=7.1 Hz, 1H, 3a′″-H), 5.15 (t, J=9.9 Hz, 1H, 6′″-H), 5.27 (dt, J=7.9 Hz/4.4 Hz, 1H, 6a′″-H), 6.95 (m, 1H, 2″-H), 7.02 (d, J=7.4 Hz, 1H, 6″-H), 7.30 (m, 1H, 5″-H). ¹³C NMR (DMSO-d₆) δ −8.9, 13.3, 17.9, 22.1, 24.7, 26.2, 26.8, 27.3, 27.4, 32.6, 35.1, 39.0, 61.8, 65.5, 66.7, 81.4, 82.0, 82.1, 82.7, 83.6, 112.1, 112.5, 122.7, 123.6, 127.9, 136.4, 144.2, 150.6, 152.4, 152.9, 154.4, 166.1, 167.9, 169.1.

### Hydroxy(tosyloxy)iodo-4-methoxybenzene (12)

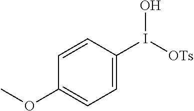 [SMILES: COc1ccc(I(O)OS(=O)(=O)c2ccc(C)cc2)cc1]

4-lodoanisole (0.12 g, 0.51 mmol) was dissolved in CH₂Cl₂. mCPBA (0.12 g, 0.70 mmol) and p-toluenesulfonic acid monohydrate (0.10 g, 0.51 mmol) were successively added and the mixture was lef to react at room temperature for 1 hour. After addition of diethylether (20 mL) and precipitation of the product, the solvents were removed by decantation. The precipitate was washed twice with diethylether (2×20 mL) and immediately engaged in the next step.

### (4-(2-((tert-butoxycarbonyl)(3-((3aS,4R,6S,6aR)-6-(2-((tert-butoxycarbonyl)oxy)ethoxy)-2,2-dimethyltetrahydro-3aH-cyclopenta[d][1,3]dioxol-4-yl)-5-(propylthio)-3H-[1,2,3]triazolo[4,5-d]pyrimidin-7-yl)amino)cyclopropyl)-2-fluorophenyl)(4-methoxyphenyl)iodonium tosylate (13)

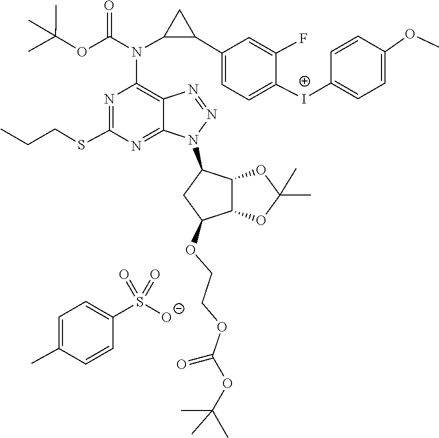 [SMILES: CCCSc1nc(N(C(=O)OC(C)(C)C)C2CC2c2ccc([I+]c3ccc(OC)cc3)c(F)c2)c2nnn([C@@H]3C[C@H](OCCOC(=O)OC(C)(C)C)[C@H]4OC(C)(C)O[C@H]43)c2n1 ; Cc1ccc(S(=O)(=O)[O-])cc1]

Hydroxy(tosyloxy)iodo-4-methoxybenzene (12) (0.14 g, 0.33 mmol) was added to a solution of (11) (0.27 g, 0.3 mmol) in dichloromethane (5 mL) at 0° C. The mixture was left to react at room temperature for 1 hour. After evaporation of the solvent, the residue was purified by silica gel column chromatography.

Yield: 65%.

Melting point: decomposition at 117° C.

¹H NMR (DMSO-d₆) δ 0.93 (q, J=7.3 Hz, 3H, SCH₂CH₂CH₃), 1.27 (s, 3H, CH₃), 1.37 (s, 9H, C(CH₃)₃), 1.39 (s, 9H, C(CH₃)₃), 1.42 (m, 1H, 3′-Ha), 1.50 (s, 3H, CH₃), 1.64 (m, 3H, SCH₂CH₂CH₃/3′-Hb), 2.29 (s, 3H, CH_{3 tos}), 2.32 (m, 1H, 2′-H), 2.56 (m, 1H, 5′″-Ha), 2.72 (m, 1H, 5′″-Hb), 2.84-3.07 (m, 2H, SCH₂CH₂CH₃), 3.30 (m, 1H, 1′-H), 3.62 (m, 2H, OCH₂CH₂OC(CH₃)₃), 3.79 (s, 3H, OCH₃), 4.04 (m, 3H, OCH₂CH₂OC(CH₃)₃/4′″-H), 4.70 (dd, J=7.2 Hz/3.0 Hz, 1H, 3a′″-H), 5.14 (m, 1H, 6′″-H), 5.25 (m, 1H, 6a′″-H), 7.07 (d, J=9.1 Hz, 2H, 3″″-H/5″″-H), 7.11 (d, J=7.8 Hz, 2H, 3-Hₜₒₛ/5-Hₜₒₛ), 7.18 (dd, J=8.4 Hz/1.7 Hz, 1H, 6″-H), 7.39 (d, J=10.0 Hz, 1H, 2″-H), 7.47 (d, J=8.1 Hz, 2H, 2-Hₜₒₛ/6-Hₜₒₛ), 8.13 (d, J=9.0 Hz, 2H, 2″″-H/6″″-H), 8.25 (dd, J=8.2 Hz/6.6 Hz, 1H, 5″-H). ¹³C NMR (DMSO-d₆) δ 13.2, 18.9, 20.8, 22.1, 24.7, 26.6, 26.8, 27.3, 27.4, 32.5, 35.1, 39.8, 55.7, 61.9, 65.5, 66.7, 81.4, 82.0, 82.8, 83.6, 100.8, 106.0, 112.5, 114.4, 117.5, 125.4, 127.8, 128.0, 136.3, 137.1, 137.5, 145.9, 149.7, 150.6, 152.2, 152.9, 154.1, 158.2, 160.2, 162.0, 169.0.

2. Preparation of the [¹⁸F]Triafluocyl:

### (Example 2:) synthesis of (1S,2S,3R,5S)-3-(7-((2-(3-fluoro-4-(fluoro-¹⁸F)phenyl)cyclopropyl)amino)-5-(propylthio)-3H-[1,2,3]triazolo[4,5-d]pyrimidin-3-yl)-5-(2-hydroxyethoxy)cyclopentane-1,2-diol (15)

### tert-butyl (3-((3aS,4R,6S,6aR)-6-(2-((tert-butoxycarbonyl)oxy)ethoxy)-2,2-dimethyltetrahydro-4H-cyclopenta[d][1,3]dioxol-4-yl)-5-(propylthio)-3H-[1,2,3]triazolo[4,5-d]pyrimidin-7-yl)(2-(3-fluoro-4-(fluoro-¹⁸F)phenyl)cyclopropyl)carbamate (14)

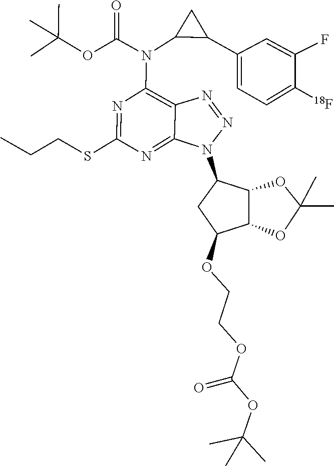 [SMILES: CCCSc1nc(N(C(=O)OC(C)(C)C)C2CC2c2ccc([18F])c(F)c2)c2nnn([C@@H]3C[C@H](OCCOC(=O)OC(C)(C)C)[C@H]4OC(C)(C)O[C@H]43)c2n1]

The radiosynthesis starts with the trapping of [¹⁸F]fluoride produced from ¹⁸O-enriched H₂O onto a carbonated QMA cartridge (46 mg). The [¹⁸F]fluoride is then transfered to the reator using a mixture of K₂₂₂ in MeOH (18 mg/mL; 675 μL) and K2CO3 in H₂O (10 mg/mL; 75 μL) as QMA eluent. After drying by azeotropic evaporation, a solution of compound 13 (0.008 g, 7 μmol) and TEMPO (0.004 g, 30 μmol) in DMF (1 mL) is added to the [¹⁸F]fluoride residue. The reactor is then closed and heated to 120-140° C. After 2-10 min, the reaction mixture is cooled down to 40° C. Compound 14 is obtained with an estimated radiochemical conversion of 17-25% as determined by TLC. The reaction mixture is used as such in the next step.

Thin Layer Chromatography identification: Stationary phase: silicagel; Mobile phase: Ethyl acetate 100%; Rf (14)=0.7-0.8; Rf ([¹⁸F]fluoride)=0.03-0.05

### (1S,2S,3R,5S)-3-(7-((2-(3-fluoro-4-(fluoro-¹⁸F)phenyl)cyclopropyl)amino)-5-(propylthio)-3H-[1,2,3]triazolo[4,5-d]pyrimidin-3-yl)-5-(2-hydroxyethoxy)cyclopentane-1,2-diol (15)

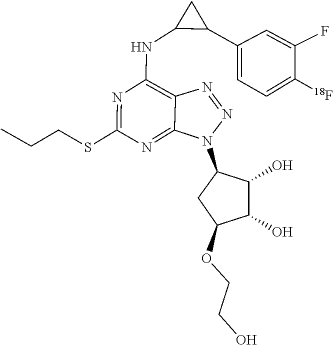 [SMILES: CCCSc1nc(NC2CC2c2ccc([18F])c(F)c2)c2nnn([C@@H]3C[C@H](OCCO)[C@@H](O)[C@H]3O)c2n1]

A solution of hydrochloric acid (0.4 mL, 4 mol/L) is added to the reaction mixture obtained in the previous step. The reactor is closed and heated to 100° C. After 1-10 min, the reaction mixture is cooled down to 40° C., neutralised by addition of NaOH (0.6 mL, 4 mol/L) and then pre-purified onto a tC18 sep-pak cartridge (400 mg). Compound 15 is obtained with a radiochemical yield of 2.5% (overall yield for the two step reaction, decay corrected) after purification of the crude product by semi-preparative HPLC onto a XSelect CSH OBD Prep Column (PN:186008239) using a mixture of H₂O and MeCN (50:50-60:40) as mobile phase (5 mL/min).

TLC identification: Stationary phase: silicagel; Mobile phase: Ethyl acetate 100%; Rf (15)=0.15-0.25.

Semi-preparative identification: RT (15)=29 min (mobile phase H₂O-MeCN 60:40)

### EXAMPLE 7

### Preparation of (1S,2R,3S,4R)-4-(7-((2-(3-fluoro-4-(fluoro¹⁸F)phenyl)cyclopropyl)amino)-5-(propylthio)-3H-[1,2,3]triazolo[4,5-d]pyrimidin-3-yl)cyclopentane-1,2,3-triol (27)

In a first step (i), (3aR,4S,6R,6aS)-6-((5-amino-6-chloro-2-(propylthio)pyrimidin-4-yl)amino)-2,2-dimethyltetrahydro-4H-cyclopenta[d][1,3]dioxol-4-ol (20) is obtained by reaction of (3aR,4S,6R,6aS)-6-amino-2,2-dimethyltetrahydro-4H-cyclopenta[d][1,3]dioxol-4-ol with 4,6-dichloro-2-(propylthio)pyrimidin-5-amine in acetonitrile at a temperature of 110° C.

This step is followed by a ring closure reaction of intermediate 20 by means of sodium nitrite carried out in acetic acid at a temperature from 5° C. to 20° C. (step ii) to obtain (3aR,4S,6R,6aS)-6-(7-chloro-5-(propylthio)-3H-[1,2,3]triazolo[4,5-d]pyrimidin-3-yl)-2,2-dimethyltetrahydro-4H-cyclopenta[d][1,3]dioxol-4-ol (21).

In step ix, (3aR,4S,6R,6aS)-6-(7-((2-(4-bromo-3-fluorophenyl)cyclopropyl)amino)-5-(propylthio)-3H-[1,2,3]triazolo[4,5-d]pyrimidin-3-yl)-2,2-dimethyltetrahydro-4H-cyclopenta[d][1,3]dioxol-4-ol (22) is obtained by nucleophilic substitution of the chlorine atom of intermediate 21 by 2-(4-bromo-3-fluorophenyl)cyclopropanamine hydrochloride in the presence of a base such as triethylamine at a temperature of 20° C.

The sensitive functions of 22 are then protected by the tert-butoxycarbonyl group (step x) after reaction of 22 with di-tert-butyl dicarbonate in tetrahydrofurane at a temperature of 20° C. to give tert-butyl (2-(4-bromo-3-fluorophenyl)cyclopropyl)(3-((3aS,4R,6S,6aR)-6-((tert-butoxycarbonyl)oxy)-2,2-dimethyltetrahydro-4H-cyclopenta[d][1,3]dioxol-4-yl)-5-(propylthio)-3H-[1,2,3]triazolo[4,5-d]pyrimidin-7-yl)carbamate (23).

The labelling position is activated in a next step (step xi) by nucleophilic substitution of the bromine atom of (23) by a trimethylstannyl group using hexamethyldistannane in the presence of tetrakis(triphenylphosphine)palladium(0) as a catalyst to give tert-butyl (3-((3aS,4R,6S,6aR)-6-((tert-butoxycarbonyl)oxy)-2,2-dimethyltetrahydro-4H-cyclopenta[d][1,3]dioxol-4-yl)-5-(propylthio)-3H-[1,2,3]triazolo[4,5-d]pyrimidin-7-yl)(2-(3-fluoro-4-(trimethylstannyl)phenyl)cyclopropyl)carbamate (24), followed by a reaction with hydroxy(tosyloxy)iodo-4-methoxybenzene (12) (step xii) to give the corresponding iodonium tosylate (25).

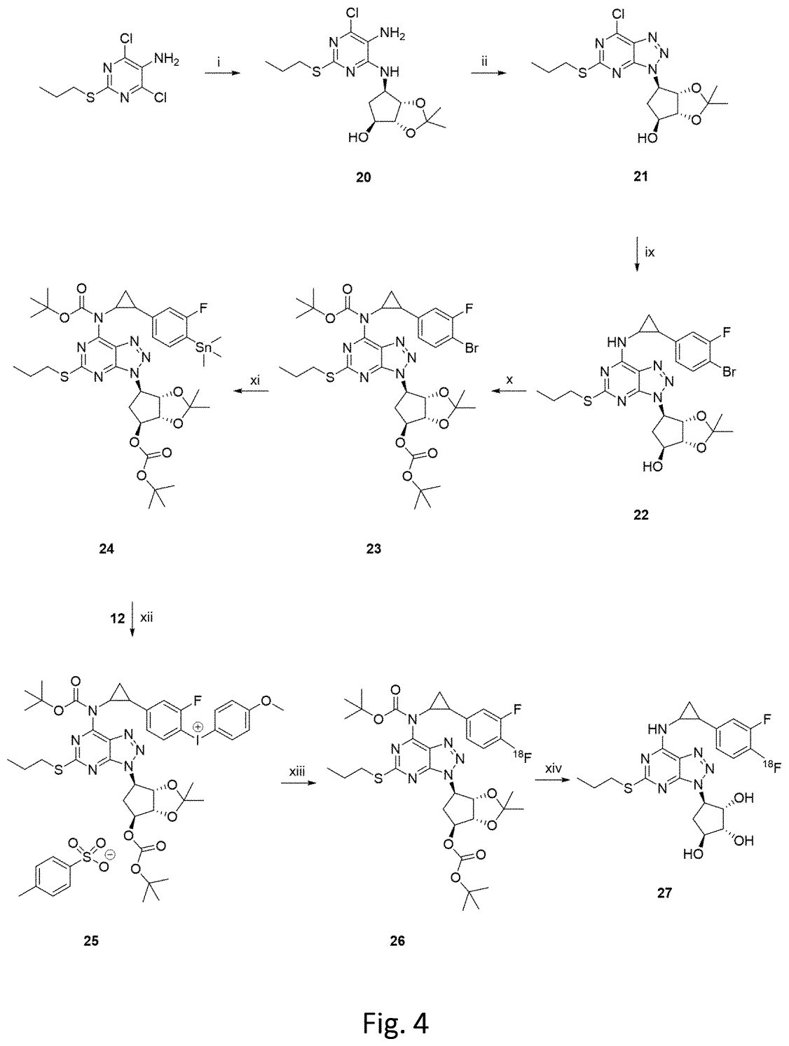

### 1. Preparation of a Labelling Precursor as Illustrated in FIG. 4

### Synthesis of (4-(2-((tert-butoxycarbonyl)(3-((3aS,4R,6S,6aR)-6-((tert-butoxycarbonyl)oxy)-2,2-dimethyltetrahydro-4H-cyclopenta[d][1,3]dioxol-4-yl)-5-(propylthio)-3H-[1,2,3]triazolo[4,5-c]pyrimidin-7-yl)amino)cyclopropyl)-2-fluorophenyl)(4-methoxyphenyl)iodonium tosylate (25)

### (3aR,4S,6R,6aS)-6-((5-amino-6-chloro-2-(propylthio)pyrimidin-4-yl)amino)-2,2-dimethyltetrahydro-4H-cyclopenta[d][1,3]dioxol-4-ol (20)

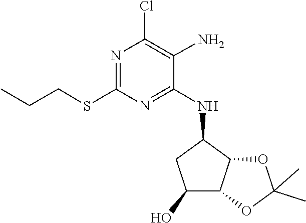 [SMILES: CCCSc1nc(Cl)c(N)c(N[C@@H]2C[C@H](O)[C@H]3OC(C)(C)O[C@H]32)n1]

The mixture of 4,6-dichloro-2-(propylthio)pyrimidin-5-amine (1.0 g, 4.2 mmol), (3aR,4S,6R,6aS)-6-amino-2,2-dimethyltetrahydrocyclopenta[d][1,3]dioxol-4-ol (0.93 g, 5.4 mmol) and triethylamine (0.6 mL, 4.2 mmol) in acetonitrile (10 mL) was introduced in a sealed vessel and heated overnight at 110° C. After evaporation of the solvent, the residue was purified by silica gel column chromatography.

Yield: 89%.

Melting point: liquid.

¹H NMR (CDCl₃) δ 1.03 (t, J=7.4 Hz, 3H, SCH₂CH₂CH₃), 1.26 (s, 3H, CH₃), 1.43 (s, 3H, CH₃), 1.76 (m, 2H, SCH₂CH₂CH₃), 1.83 (d, J=14.5 Hz, 1H, 5′-Ha), 2.14 (s, 1H, OH), 2.36 (m, 1H, 5′-Hb), 3.01 (ddd, J=13.4 Hz/8.3 Hz/6.4 Hz, 1H, SCHa), 3.09 (s, 2H, NH₂), 3.13 (ddd, J=13.5 Hz/8.3 Hz/6.4 Hz, 1H, SCHb), 4.38 (s, 1H, 4′-H), 4.51 (dd, J=5.4 Hz/1.7 Hz, 1H, 3a′-H), 4.57 (dd, J=5.4 Hz/1.2 Hz, 1H, 6a′-H), 4.62 (t, J=7.4 Hz, 1H, 6′-H), 5.93 (d, J=8.4 Hz, 1H, NH). ¹³C NMR (CDCl₃) δ 13.5, 23.2, 23.8, 26.2, 33.2, 35.0, 57.2, 78.1, 85.2, 86.2, 110.3, 117.2, 144.4, 154.5, 162.0.

### (3aR,4S,6R,6aS)-6-(7-chloro-5-(propylthio)-3H-[1,2,3]triazolo[4,5-d]pyrimidin-3-yl)-2,2-dimethyltetrahydro-4H-cyclopenta[d][1,3]dioxol-4-ol (21)

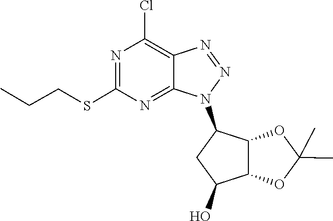 [SMILES: CCCSc1nc(Cl)c2nnn([C@@H]3C[C@H](O)[C@H]4OC(C)(C)O[C@H]43)c2n1]

To a solution of (20) (1.0 g, 2.67 mmol) in acetic acid (10 mL) cooled on an ice bath was added NaNO₂ (250 mg, 3.56 mmol). The mixture was allowed to reach room temperature within 1 hour and water (40 mL) was then added. The resulting mixture was extracted with ethyl acetate (3×50 mL) and the combined organic layers were dried over MgSO₄ and evaporated to give an oily residue.

Yield: 85%.

Melting point: oil.

¹H NMR (CDCl₃) δ 1.10 (t, J=7.4 Hz, 3H, SCH₂CH₂CH₃), 1.33 (s, 3H, CH₃), 1.54 (s, 3H, CH₃), 1.83 (h, J=7.3 Hz, 2H, SCH₂CH₂CH₃), 2.38 (d, J=15.3 Hz, 1H, 5′-Ha), 2.90 (m, 1H, 5′-Hb), 3.24 (td, J=7.1 Hz/1.7 Hz, 2H, SCH₂CH₂CH₃), 3.76 (d, J=8.8 Hz, 1H, OH), 4.44 (m, 1H, 4′-H), 4.80 (d, J=5.6 Hz, 1H, 3a′-H), 5.04 (d, J=5.7 Hz, 1H, 6a′-H), 5.34 (d, J=7.7 Hz, 1H, 6′-H). ¹³C NMR (CDC1₃) 5 13.5, 22.1, 24.1, 26.6, 33.8, 37.0, 64.1, 76.7, 85.3, 87.6, 111.6, 132.0, 150.2, 153.7, 172.5.

### (3aR,4S,6R,6aS)-6-(7-((2-(4-bromo-3-fluorophenyl)cyclopropyl)amino)-5-(propylthio)-3H-[1,2,3]triazolo[4,5-d]pyrimidin-3-yl)-2,2-dimethyltetrahydro-4H-cyclopenta[d][1,3]dioxol-4-ol (22)

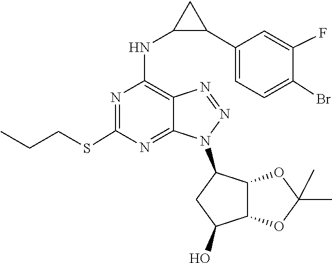 [SMILES: CCCSc1nc(NC2CC2c2ccc(Br)c(F)c2)c2nnn([C@@H]3C[C@H](O)[C@H]4OC(C)(C)O[C@H]43)c2n1]

The mixture of (21) (0.5 g, 1.30 mmol), 2-(4-bromo-3-fluorophenyl)cyclopropanamine hydrochloride (0.34 g, 1.30 mmol) and triethylamine (0.22 mL, 1.50 mmol) in acetonitrile (10 mL) was left to react at room temperature for 2 hours. After evaporation of the solvent, the residue was purified by silica gel column chromatography.

Yield: 67%.

Melting point: 136-139° C.

¹H NMR (DMSO-d₆) δ 0.82 (td, J=7.3 Hz/2.6 Hz, 2.4H, SCH₂CH₂CH₃ major), 1.00 (t, J=7.3 Hz, 0.6H, SCH₂CH₂CH₃ minor), 1.25 (s, 3H, CH₃), 1.42 (m, 1H, 3′-Ha), 1.46 (s, 3H, CH₃), 1.50 (m, 1.8H, 3′-Hb minor/SCH₂CH₂CH₃ major), 1.59 (m, 0.8H, 3′-Hb major), 1.71 (h, J=7.3 Hz, 0.4H, SCH₂CH₂CH₃ minor), 2.13 (m, 0.8H, 2′-H major), 2.24 (m, 0.2H, 2′-H minor), 2.44 (m, 1H, 5′″-Ha), 2.53 (m, 1H, 5′″-Hb), 2.85 (m, 0.8H, SCHₐCH₂CH₃ major), 2.91 (m, 0.8H, SCH_{b}CH₂CH₃ major), 3.08 (m, 0.4H, SCH₂CH₂CH₃ minor), 3.18 (m, 0.8H, 1′-H major), 3.78 (m, 0.2H, 1′-H minor), 4.13 (m, 1H, 4′″-H), 4.54 (m, 1H, 3a′″-H), 4.98 (m, 1H, 6′″-H), 5.21 (m, 0.2H, 6a′″-H minor), 5.26 (m, 1.8H, 6a′″-H major/OH), 6.97 (d, J=7.2 Hz, 0.2H, 6″-H minor), 7.03 (d, J=8.3 Hz, 0.8H, 6″-H major), 7.18 (d, J=9.0 Hz, 0.2H, 2″-H minor), 7.25 (d, J=10.4 Hz, 0.8H, 2″-H major), 7.59 (t, J=7.9 Hz, 1H, 5″-H), 9.01 (d, J=3.1 Hz, 0.2H, NH minor), 9.40 (s, 0.8H, NH major). ¹³C NMR (DMSO-d₆) δ 13.0, 15.3, 22.3, 24.3, 24.7, 26.9, 32.4, 34.5, 37.8, 61.8, 74.2, 82.1, 86.1, 104.5, 111.9, 114.0, 123.2, 123.9, 133.0, 144.3, 149.0, 153.9, 157.6, 159.0, 169.4.

### tert-butyl (2-(4-bromo-3-fluorophenyl)cyclopropyl)(3-((3aS,4R,6S,6aR)-6-((tert-butoxycarbonyl)oxy)-2,2-dimethyltetrahydro-4H-cyclopenta[d][1,3]dioxol-4-yl)-5-(propylthio)-3H-[1,2,3]triazolo[4,5-d]pyrimidin-7-yl)carbamate (23)

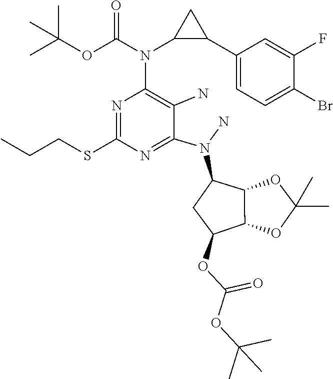

The mixture of (22) (0.58 g, 1.0 mmol), di-tert-butyl dicarbonate (1 g, 4.6 mmol), and 4-(dimethylamino)pyridine (30 mg, cat.) in tetrahydrofurane (10 mL) was left to react overnight at room temperature. After evaporation of the solvent, the residue was purified by silica gel column chromatography.

Yield: 91%.

Melting point: 90-98° C.

¹H NMR (DMSO-d₆) δ 0.98 (td, J=7.3 Hz/3.2 Hz, 3H, SCH₂CH₂CH₃), 1.31 (s, 3H, CH₃), 1.32 (m, 1H, 3′-Ha), 1.33 (s, 9H, C(CH₃)₃), 1.40 (s, 9H, C(CH₃)₃), 1.51 (s, 3H, CH₃), 1.56 (q, J=6.6 Hz, 1H, 3′-Hb), 1.70 (m, 2H, SCH₂CH₂CH₃), 2.25 (m, 1H, 2′-H), 2.73-2.84 (m, 2H, 5′″-CH₂), 3.02 (m, 1H, SCHaCH₂CH₃), 3.11 (m, 1H, SCHbCH₂CH₃), 3.27 (m, 1H, 1′-H), 4.80 (t, J=5.4 Hz, 1H, 3a′″-H), 4.91 (t, J=5.0 Hz, 1H, 4′″-H), 5.25 (m, 1H, 6′″-H), 5.50 (m, 1H, 6a′″-H), 7.03 (ddd, J=7.7 Hz/5.1 Hz/1.9 Hz, 1H, 6″-H), 7.24 (ddd, J=10.1 Hz/6.9 Hz/1.9 Hz, 1H, 2″-H), 7.59 (t, J=7.9 Hz, 1H, 5″-H). ¹³C NMR (DMSO-d₆) δ 13.3, 18.1, 22.0, 24.4, 26.1, 26.5, 27.2, 27.4, 32.6, 33.9, 34.0, 62.8, 79.8, 81.8, 82.0, 82.7, 83.5, 104.9, 111.7, 114.7, 124.3, 127.8, 132.9, 143.2, 150.8, 151.9, 152.3, 154.2, 157.4, 158.8, 169.0.

### tert-butyl (3-((3aS,4R,6S,6aR)-6-((tert-butoxycarbonyl)oxy)-2,2-dimethyltetrahydro-4H-cyclopenta[d][1,3]dioxol-4-yl)-5-(propylthio)-3H-[1,2,3]triazolo[4,5-d]pyrimidin-7-yl)(2-(3-fluoro-4-(trimethylstannyl)phenyl)cyclopropyl)carbamate (24)

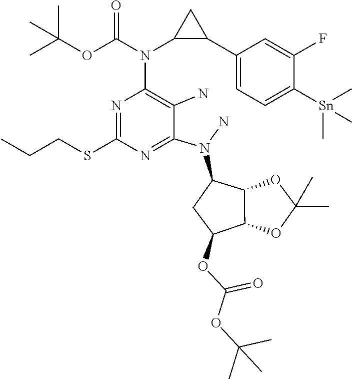

The mixture of (23) (0.39 g, 0.5 mmol), hexamethyldistannane (0.49 g, 1.5 mmol), and tetrakis(triphenylphosphine)palladium(0) (10 mg, cat.) in dry toluene (5 mL) was introduced in a sealed vessel and heated overnight at 100° C. under nitrogen. After cooling, ethyl acetate (50 mL) was added and the insoluble was filtered off. The filtrate was evaporated to dryness and the resulting residue was purified by silica gel column chromatography.

Yield: 72%.

Melting point: 105-110° C.

¹H NMR (DMSO-d₆) δ 0.30 (s, 9H, Sn(CH₃)₃), 0.96 (td, J=7.3 Hz/3.6 Hz, 3H, SCH₂CH₂CH₃), 1.28 (m, 1H, 3′-Ha), 1.31 (s, 3H, CH₃), 1.33 (s, 9H, C(CH₃)₃), 1.40 (s, 9H, C(CH₃)₃), 1.51 (m, 4H, CH₃/3′-Hb), 1.68 (m, 2H, SCH₂CH₂CH₃), 2.22 (m, 1H, 2′-H), 2.73-2.84 (m, 2H, 5′″-CH₂), 2.98 (m, 1H, SCHaCH₂CH₃), 3.08 (m, 1H, SCHbCH₂CH₃), 3.24 (dt, J=7.0 Hz/3.5 Hz, 1H, 1′-H), 4.80 (m, 1H, 3a″-H), 4.91 (t, J=4.9 Hz, 1H, 4′″-H), 5.24 (dq, J=8.7 Hz/4.3 Hz/2.6 Hz, 1H, 6′″-H), 5.51 (dt, J=6.3 Hz/2.5 Hz, 1H, 6a′″-H), 6.95 (m, 1H, 2″-H), 7.02 (dd, J=7.4 Hz/3.4 Hz, 1H, 6″-H), 7.30 (m, 1H, 5″-H). ¹³C NMR (DMSO-d₆) δ −8.9, 13.3, 17.9, 22.0, 24.4, 26.3, 26.5, 27.2, 27.4, 32.6, 33.9, 34.0, 62.8, 79.9, 81.8, 82.0, 82.1, 82.7, 83.5, 111.7, 112.2, 122.8, 123.6, 123.9, 127.8, 136.4, 144.1, 150.8, 151.9, 152.4, 154.3, 166.3, 167.7, 169.1.

### (4-(2-((tert-butoxycarbonyl)(3-((3aS,4R,6S,6aR)-6-((tert-butoxycarbonyl)oxy)-2,2-dimethyltetrahydro-4H-cyclopenta[d][1,3]dioxol-4-yl)-5-(propylthio)-3H-[1,2,3]triazolo[4,5-d]pyrimidin-7-yl)amino)cyclopropyl)-2-fluorophenyl)(4-methoxyphenyl)iodonium tosylate (25)

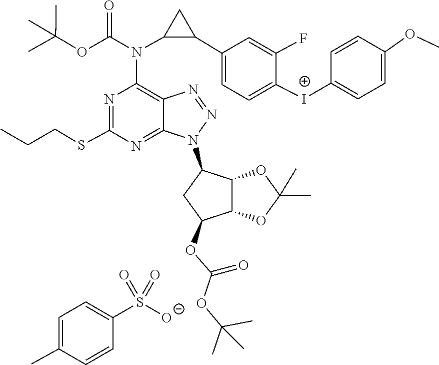

Hydroxy(tosyloxy)iodo-4-methoxybenzene (12) (0.14 g, 0.33 mmol) was added to a solution of (24) (0.26 g, 0.3 mmol) in dichloromethane (5 mL) at 0° C. The mixture was left to react at room temperature for 1 hour. After evaporation of the solvent, the residue was purified by silica gel column chromatography.

Yield: 47%.

Melting point: decomposition at 124° C.

¹H NMR (DMSO-d₆) δ 0.93 (q, J=7.4 Hz, 3H, SCH₂CH₂CH₃), 1.30 (s, 3H, CH₃), 1.32 (s, 9H, C(CH₃)₃), 1.37 (s, 9H, C(CH₃)₃), 1.39 (m, 1H, 3′-Ha), 1.50 (s, 3H, CH₃), 1.64 (m, 3H, SCH₂CH₂CH₃/3′-Hb), 2.29 (s, 3H, CH_{3 tos}), 2.31 (m, 1H, 2′-H), 2.71-2.84 (m, 2H, 5′″-CH₂), 2.91 (m, 1H, SCHaCH₂CH₃), 3.03 (m, 1H, SCHbCH₂CH₃), 3.30 (m, 1H, 1′-H), 3.79 (s, 3H, OCH₃), 4.79 (d, J=4.8 Hz, 1H, 3a′″-H), 4.91 (m, 1H, 4′″-H), 5.23 (m, 1H, 6′″-H), 5.48 (m, 1H, 6a′″-H), 7.06 (d, J=9.2 Hz, 2H, 3″″-H/5″″-H), 7.11 (d, J=7.8 Hz, 2H, 3-Hₜₒₛ/5-Hₜₒₛ), 7.18 (ddd, J=8.4 Hz/4.4 Hz/1.7 Hz, 1H, 6″-H), 7.38 (ddd, J=9.8 Hz/6.2 Hz/1.7 Hz, 1H, 2″-H), 7.47 (d, J=8.0 Hz, 2H, 2-Hₜₒₛ/6-Hₜₒₛ), 8.12 (d, J=9.1 Hz, 2H, 2″″-H/6″″-H), 8.25 (dd, J=8.2 Hz/6.5 Hz, 1H, 5″-H). ¹³C NMR (DMSO-d₆) δ 13.2, 18.8, 20.8, 22.1, 24.4, 26.5, 26.7, 27.2, 27.4, 29.6, 32.5, 33.9, 34.0, 55.7, 55.8, 62.8, 79.8, 81.8, 82.0, 82.9, 83.5, 101.1, 106.2, 111.8, 114.3, 117.5, 125.5, 127.7, 128.0, 136.3, 137.1, 137.5, 145.9, 149.6, 150.8, 151.8, 152.2, 154.1, 158.5, 159.9, 161.9, 169.0.

### 2. Preparation of (1S,2R,3S,4R)-4-(7-((2-(3-fluoro-4-(fluoro¹⁸F)phenyl)cyclopropyl)amino)-5-(propylthio)-3H-[1,2,3]triazolo[4,5-d]pyrimidin-3-yl)cyclopentane-1,2,3-triol as Defined in Formula (III) (27)

### Synthesis of tert-butyl (3-((3aS,4R,6S,6aR)-6-((tert-butoxycarbonyl)oxy)-2,2-dimethyltetrahydro-4H-cyclopenta[d][1,3]dioxol-4-yl)-5-(propylthio)-3H-[1,2,3]triazolo[4,5-d]pyrimidin-7-yl)(2-(3-fluoro-4-(fluoro-¹⁸F)phenyl)cyclopropyl)carbamate (26)

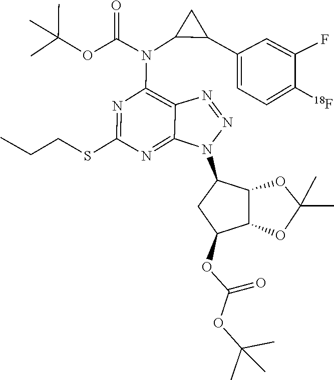 [SMILES: CCCSc1nc(N(C(=O)OC(C)(C)C)C2CC2c2ccc([18F])c(F)c2)c2nnn([C@@H]3C[C@H](OC(=O)OC(C)(C)C)[C@H]4OC(C)(C)O[C@H]43)c2n1]

The radiosynthesis starts with the trapping of [¹⁸F]fluoride produced from ¹⁸O-enriched H₂O onto a carbonated QMA cartridge (46 mg). The [¹⁸F]fluoride is then transfered to the reactor using a mixture of K₂₂₂ in MeOH (18 mg/mL; 675 μL) and K₂CO₃ in H₂O (10 mg/mL; 75 μL) as QMA eluent. After drying by azeotropic evaporation, a solution of compound 25 (0.008 g, 7 μmol) and TEMPO (0.004 g, 30 μmol) in DMF (1 mL) is added to the [¹⁸F]fluoride residue. The reactor is then closed and heated to 120-140° C. After 2-10 min, the reaction mixture is cooled down to 40° C. Compound 26 is obtained with an estimated radiochemical conversion of 5-10% as determined by Thin Layer Chromatography (TLC). The reaction mixture is used as such in the next step.

TLC identification: Stationary phase: silicagel; Mobile phase: Ethyl acetate 100%; Retention factor Rf (26)=0.8-0.9; Retention factor Rf ([¹⁸F]fluoride)=0.03-0.05

### (1S,2R,3S,4R)-4-(7-((2-(3-fluoro-4-(fluoro-¹⁸F)phenyl)cyclopropyl)amino)-5-(propylthio)-3H-[1,2,3]triazolo[4,5-d]pyrimidin-3-yl)cyclopentane-1,2,3-triol (27)

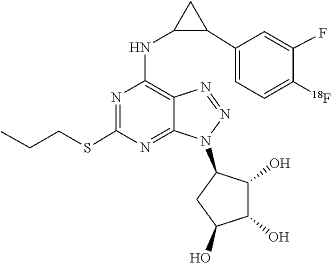 [SMILES: CCCSc1nc(NC2CC2c2ccc([18F])c(F)c2)c2nnn([C@@H]3C[C@H](O)[C@@H](O)[C@H]3O)c2n1]

A solution of hydrochloric acid (0.4 mL, 4 mol/L) is added to the reaction mixture obtained in the previous step. The reactor is closed and heated to 100° C. After 1-10 min, the reaction mixture is cooled down to 40° C., neutralised by addition of NaOH (0.6 mL, 4 mol/L) and then pre-purified onto a tC18 sep-pak cartridge (400 mg). Compound 27 is obtained with a radiochemical yield of 1 to 2% (overall yield for the two step reaction, decay corrected) after purification of the crude product by semi-preparative HPLC onto a XSelect CSH OBD Prep Column (PN:186008239) using a mixture of H₂O and MeCN (50:50-60:40) as mobile phase (5 mL/min).

TLC identification: Stationary phase: silicagel; Mobile phase: Ethyl acetate 100%; Rf (27)=0.2-0.3.

Semi-preparative identification: Retention Time (27)=40 min (mobile phase H₂O-MeCN 60:40).

What is claimed is:

1. A method of imaging a bacterial infection in a host mammal comprising:
(a) administering the the host mammal an effective amount of triazolo[4,5-d]pyrimidine derivative of formula (I): 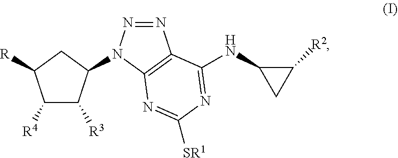
wherein R¹ is C₃₋₅ alkyl optionally substituted by one or more halogen atoms; R² is a phenyl group, optionally substituted by one or more halogen atoms; R³ and R⁴ are each hydroxyl; R is XOH, wherein X is CH₂, OCH₂CH₂, or a bond;
or a pharmaceutical acceptable salt or solvate thereof, or a solvate of such a salt provided that when X is CH₂ or a bond, R¹ is not propyl; when X is CH₂ and R¹ CH₂CH₂CF₃, butyl or pentyl, the phenyl group at R² must be substituted by fluorine; when X is OCH₂CH₂ and R¹ is propyl, the phenyl group at R² must be substituted by fluorine;
and wherein the triazolo[4,5-d]pyrimidine derivative of formula (I) comprises or is bound to a detectable marker; and
(b) tracking said detectable triazolo[4,5-d]pyrimidine derivative by an imaging technique to display the bacterial infection.

2. The method according to claim 1, wherein the triazolo[4,5-d]pyrimidine derivative of formula (I) is bound to a transporter comprising a detectable marker.

3. The method according to claim 1, wherein the detectable marker is a signal amplifier.

4. The method according to claim 3, wherein the transporter is a micelle, a microsphere, a liposome, a nanosphere, a nanosuspension, a nanoemulsion, or a nanocapsule.

5. The method according to claim 1, wherein the detectable marker is one or more of ²H, ³H, ¹³F^{,18}F, ¹⁹F, ¹¹C, ¹³C, ¹⁴C, ⁷⁵Br, ⁷⁶Br, ¹²⁰I, ¹²³I, ¹²⁵I, ¹³¹I, ¹⁵O, ¹³N, and/or ⁷⁸Br.

6. The method according to claim 1, wherein the detectable maker is one or more of ⁹⁹Tc, ¹²³I or ¹¹¹IN.

7. The method according to claim 1, wherein R₂ is phenyl substituted by fluorine atoms.

8. The method according to claim 1, wherein R is OH or OCH₂ CH₂OH, preferably R is OH.

9. The method according to claim 1 wherein the triazolo[4,5-d]pyrimidine derivative is selected from the group consisting of:
(1R-(1α, 2α, 3β(1R*, 2*),5β))-3-(7-((2-(3,4-difluorophenyl)cyclopropyl)amino)-5-((3,3,3-trifluoropropyl)thio)-3H-1,2,3-triazolo[4,5-d]pyrimidine-3-yl)-5-(hydroxy)cyclopentane-1,2-diol;
(1S-(1α, 2α, 3β(1R*, 2*),5β))-3-(7-((2-(3,4-difluorophenyl)cyclopropyl)amino)-5-(propylthio)-3H-1,2,3-triazolo[4,5-d]pyrimidin-3-yl)-5-(2-hydroxyethoxy)cyclopentane-1,2-diol;
(1S,2S,3R,5S)-3-[7-[(1R,2S)-2-(3,4-difluorophenyl)cyclopropylamino]-5-(propylthio)-3H-[1,2,3]triazolo[4,5-d]pyrimidin-3-yl]-5-(2-hydroxyethoxy)-1,2-cyclopentanediol);
(1S,2S,3R,5S)-3-[7-[(1R,2S)-2-(4-fluorophenyl)cyclopropylamino]-5-(propylthio)-3H-[1,2,3]triazolo[4,5-d]pyrimidin-3-yl]-5-(2-hydroxyethoxy)-1,2-cyclopentanediol);
1S,2R,3S,4R)-4-[7-[[(1R,2S)-2-(3,4-Difluorophenyl)cyclopropyl]amino]-5-(propylthio)-3H-1,2,3-triazolo[4,5-d]pyrimidin-3-yl]-1,2,3-cyclopentanetriol; and
a pharmaceutical acceptable salt or solvate thereof, or a solvate thereof or a solvate of such a salt.

10. The method according to claim 1, wherein the triazolo[4,5-d]pyrimidine derivative is (1S,2S,3R,5S)-3-[7-[(1R,2S)-2-(3,4-difluorophenyl)cyclopropylamino]-5 -(propylthio)-3H-[1,2,3]triazolo[4,5-d]pyrimidin-3-yl]-5-(2-hydroxyethoxy)-1,2-cyclopentanediol) also called Triafluocyl.

11. The method according to claim 1, wherein the triazolo[4,5-d]pyrimidine derivative is 1S,2R,3S,4R)-4-[7-[[(1R,2S)-2-(3,4-Difluorophenyl)cyclopropyl]amino]-5-(propylthio)-3H-1,2,3-triazolo[4,5-d]pyrimidin-3-yl]-1,2,3-cyclopentanetriol also called Fluometacyl.

12. The method according to claim 1, wherein the bacterial infection is caused by one or more bacteria selected from the group consisting of S. aureus, S. epidermidis, E. faecalis, E. faecium, methicillin-resistant S. aureus (MRSA), methicillin-resistant S. epidermidis (MRSE), glycopeptide intermediate S. aureus (GISA), Coagulase-negative staphylococci (CoNS), Vancomycin-resistant enterococci (VRE), beta-hemolytic Streptococcus agalactiae (Group B Streptococcus, GBS), and other streptococci.

13. The method according to claim 1, wherein the bacterial infection is caused by one or more bacteria selected from the group consisting of Acinetobacter baumannil, Pseudomonas aeruginosa, carbapenem-resistant Pseudomonas aeruginosa, Enterobacteriaceae, and 3^{rd} generation cephalosporin-resistant Enterobacteriaceae (Klebsiella pneumonia, Escherichia coli, Enterobacter spp, Serratia spp, Proteus spp, Providentia spp, and Morganella spp).

14. A pharmaceutical composition for diagnosing and/or prognosing in-vivo bacterial infection in a host mammal comprising:
(a) a triazolo[4,5-d]pyrimidine derivative of formula (I): 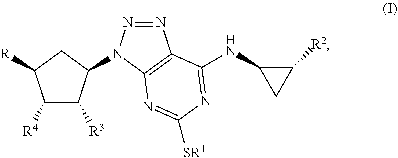
wherein R¹ is C₃₋₅ alkyl optionally substituted by one or more halogen atoms; R² is a phenyl group, optionally substituted by one or more halogen atoms; R³ and R⁴ are each hydroxyl; R is XOH, wherein X is CH₂, OCH₂CH₂, or a bond;
or a pharmaceutical acceptable salt or solvate thereof, or a solvate of such a salt provided that when X is CH₂ or a bond, R¹ is not propyl; when X is CH₂ and R¹ CH₂CH₂CF₃, butyl or pentyl, the phenyl group at R² must be substituted by fluorine; when X is OCH₂CH₂ and R¹ is propyl, the phenyl group at R² must be substituted by fluorine;
wherein the triazolo [4,5 -d]pyrimidine derivative of formula (I) comprises or is bound to a detectable marker; and
(b) a pharmaceutically acceptable additive.

15. A tracer for prognosis and/or diagnosis of bacterial infection in a host mammal, comprising a Triazolo(4,5-d)pyrimidine derivative of formula (I) 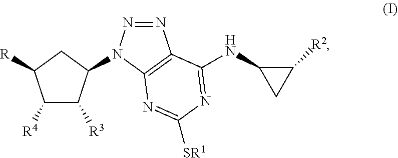
wherein R¹ is C₃₋₅ alkyl optionally substituted by one or more halogen atoms; R² is a phenyl group, optionally substituted by one or more halogen atoms; R³ and R⁴ are each hydroxyl; R is XOH, wherein X is CH₂, OCH₂CH₂, or a bond;
or a pharmaceutical acceptable salt or solvate thereof, or a solvate of such a salt provided that when X is CH₂ or a bond, R¹ is not propyl; when X is CH₂ and R¹ CH₂CH₂CF3, butyl or pentyl, the phenyl group at R² must be substituted by fluorine; when X is OCH₂CH₂ and R¹ is propyl, the phenyl group at R² must be substituted by fluorine; and
wherein the triazolo[4,5-d]pyrimidine derivative of formula (I) comprises or is bound to a detectable marker.

16. The method of claim 1, wherein the imaging technique to display the bacterial infection is single-photon emission computer tomography (SPECT).
